SEQUENCE LISTING (198 sequences)

SEQ ID NO: 1 - protein, 13 aa, synthetic construct
EWMGIINPRGDGT

SEQ ID NO: 2 - protein, 14 aa, synthetic construct
EWMGIINPRGDGTR

SEQ ID NO: 3 - protein, 15 aa, synthetic construct
EWMGIINPRGDGTRY

SEQ ID NO: 4 - protein, 16 aa, synthetic construct
EWMGIINPRGDGTRYA

SEQ ID NO: 5 - protein, 17 aa, synthetic construct
EWMGIINPRGDGTRYAQ

SEQ ID NO: 6 - protein, 19 aa, synthetic construct
EWMGIINPRGDGTRYAQKF

SEQ ID NO: 7 - protein, 18 aa, synthetic construct
EWMGIINPRGDGTRYAQK

SEQ ID NO: 8 - protein, 20 aa, synthetic construct
EWMGIINPRGDGTRYAQKFQ

SEQ ID NO: 9 - protein, 21 aa, synthetic construct
EWMGIINPRGDGTRYAQKFQG

SEQ ID NO: 10 - protein, 22 aa, synthetic construct
EWMGIINPRGDGTRYAQKFQGR

SEQ ID NO: 11 - protein, 23 aa, synthetic construct
EWMGIINPRGDGTRYAQKFQGRV

SEQ ID NO: 12 - protein, 24 aa, synthetic construct
EWMGIINPRGDGTRYAQKFQGRVT

SEQ ID NO: 13 - protein, 25 aa, synthetic construct
EWMGIINPRGDGTRYAQKFQGRVTM

SEQ ID NO: 14 - protein, 26 aa, synthetic construct
EWMGIINPRGDGTRYAQKFQGRVTMT

SEQ ID NO: 15 - protein, 7 aa, synthetic construct
GDGTRYA

SEQ ID NO: 16 - protein, 8 aa, synthetic construct
GDGTRYAQ

SEQ ID NO: 17 - protein, 9 aa, synthetic construct
GDGTRYAQK

SEQ ID NO: 18 - protein, 10 aa, synthetic construct
GDGTRYAQKF

SEQ ID NO: 19 - protein, 11 aa, synthetic construct
GDGTRYAQKFQ

SEQ ID NO: 20 - protein, 12 aa, synthetic construct
GDGTRYAQKFQG

SEQ ID NO: 21 - protein, 13 aa, synthetic construct
GDGTRYAQKFQGR

SEQ ID NO: 22 - protein, 14 aa, synthetic construct
GDGTRYAQKFQGRV

SEQ ID NO: 23 - protein, 15 aa, synthetic construct
GDGTRYAQKFQGRVT

SEQ ID NO: 24 - protein, 16 aa, synthetic construct
GDGTRYAQKFQGRVTM

SEQ ID NO: 25 - protein, 17 aa, synthetic construct
GDGTRYAQKFQGRVTMT

SEQ ID NO: 26 - protein, 9 aa, synthetic construct
GIINPRGDG

SEQ ID NO: 27 - protein, 10 aa, synthetic construct
GIINPRGDGT

SEQ ID NO: 28 - protein, 11 aa, synthetic construct
GIINPRGDGTR

SEQ ID NO: 29 - protein, 12 aa, synthetic construct
GIINPRGDGTRY

SEQ ID NO: 30 - protein, 13 aa, synthetic construct
GIINPRGDGTRYA

SEQ ID NO: 31 - protein, 14 aa, synthetic construct
GIINPRGDGTRYAQ

SEQ ID NO: 32 - protein, 15 aa, synthetic construct
GIINPRGDGTRYAQK

SEQ ID NO: 33 - protein, 16 aa, synthetic construct
GIINPRGDGTRYAQKF

SEQ ID NO: 34 - protein, 17 aa, synthetic construct
GIINPRGDGTRYAQKFQ

SEQ ID NO: 35 - protein, 18 aa, synthetic construct
GIINPRGDGTRYAQKFQG

SEQ ID NO: 36 - protein, 19 aa, synthetic construct
GIINPRGDGTRYAQKFQGR

SEQ ID NO: 37 - protein, 20 aa, synthetic construct
GIINPRGDGTRYAQKFQGRV

SEQ ID NO: 38 - protein, 21 aa, synthetic construct
GIINPRGDGTRYAQKFQGRVT

SEQ ID NO: 39 - protein, 22 aa, synthetic construct
GIINPRGDGTRYAQKFQGRVTM

SEQ ID NO: 40 - protein, 23 aa, synthetic construct
GIINPRGDGTRYAQKFQGRVTMT

SEQ ID NO: 41 - protein, 16 aa, synthetic construct
GLEWMGIINPRGDGTR

SEQ ID NO: 42 - protein, 17 aa, synthetic construct
GLEWMGIINPRGDGTRY

SEQ ID NO: 43 - protein, 18 aa, synthetic construct
GLEWMGIINPRGDGTRYA

SEQ ID NO: 44 - protein, 19 aa, synthetic construct
GLEWMGIINPRGDGTRYAQ

SEQ ID NO: 45 - protein, 20 aa, synthetic construct
GLEWMGIINPRGDGTRYAQK

SEQ ID NO: 46 - protein, 21 aa, synthetic construct
GLEWMGIINPRGDGTRYAQKF

SEQ ID NO: 47 - protein, 22 aa, synthetic construct
GLEWMGIINPRGDGTRYAQKFQ

SEQ ID NO: 48 - protein, 23 aa, synthetic construct
GLEWMGIINPRGDGTRYAQKFQG

SEQ ID NO: 49 - protein, 24 aa, synthetic construct
GLEWMGIINPRGDGTRYAQKFQGR

SEQ ID NO: 50 - protein, 25 aa, synthetic construct
GLEWMGIINPRGDGTRYAQKFQGRV

SEQ ID NO: 51 - protein, 26 aa, synthetic construct
GLEWMGIINPRGDGTRYAQKFQGRVT

SEQ ID NO: 52 - protein, 18 aa, synthetic construct
GQGLEWMGIINPRGDGTR

SEQ ID NO: 53 - protein, 19 aa, synthetic construct
GQGLEWMGIINPRGDGTRY

SEQ ID NO: 54 - protein, 20 aa, synthetic construct
GQGLEWMGIINPRGDGTRYA

SEQ ID NO: 55 - protein, 21 aa, synthetic construct
GQGLEWMGIINPRGDGTRYAQ

SEQ ID NO: 56 - protein, 22 aa, synthetic construct
GQGLEWMGIINPRGDGTRYAQK

SEQ ID NO: 57 - protein, 23 aa, synthetic construct
GQGLEWMGIINPRGDGTRYAQKF

SEQ ID NO: 58 - protein, 7 aa, synthetic construct
INPRGDG

SEQ ID NO: 59 - protein, 8 aa, synthetic construct
INPRGDGT

SEQ ID NO: 60 - protein, 9 aa, synthetic construct
INPRGDGTR

SEQ ID NO: 61 - protein, 10 aa, synthetic construct
INPRGDGTRY

SEQ ID NO: 62 - protein, 11 aa, synthetic construct
INPRGDGTRYA

SEQ ID NO: 63 - protein, 12 aa, synthetic construct
INPRGDGTRYAQ

SEQ ID NO: 64 - protein, 13 aa, synthetic construct
INPRGDGTRYAQK

SEQ ID NO: 65 - protein, 14 aa, synthetic construct
INPRGDGTRYAQKF

SEQ ID NO: 66 - protein, 15 aa, synthetic construct
INPRGDGTRYAQKFQ

SEQ ID NO: 67 - protein, 16 aa, synthetic construct
INPRGDGTRYAQKFQG

SEQ ID NO: 68 - protein, 17 aa, synthetic construct
INPRGDGTRYAQKFQGR

SEQ ID NO: 69 - protein, 18 aa, synthetic construct
INPRGDGTRYAQKFQGRV

SEQ ID NO: 70 - protein, 19 aa, synthetic construct
INPRGDGTRYAQKFQGRVT

SEQ ID NO: 71 - protein, 20 aa, synthetic construct
INPRGDGTRYAQKFQGRVTM

SEQ ID NO: 72 - protein, 21 aa, synthetic construct
INPRGDGTRYAQKFQGRVTMT

SEQ ID NO: 73 - protein, 8 aa, synthetic construct
IINPRGDG

SEQ ID NO: 74 - protein, 9 aa, synthetic construct
IINPRGDGT

SEQ ID NO: 75 - protein, 10 aa, synthetic construct
IINPRGDGTR

SEQ ID NO: 76 - protein, 11 aa, synthetic construct
IINPRGDGTRY

SEQ ID NO: 77 - protein, 12 aa, synthetic construct
IINPRGDGTRYA

SEQ ID NO: 78 - protein, 13 aa, synthetic construct
IINPRGDGTRYAQ

SEQ ID NO: 79 - protein, 14 aa, synthetic construct
IINPRGDGTRYAQK

SEQ ID NO: 80 - protein, 15 aa, synthetic construct
IINPRGDGTRYAQKF

SEQ ID NO: 81 - protein, 16 aa, synthetic construct
IINPRGDGTRYAQKFQ

SEQ ID NO: 82 - protein, 17 aa, synthetic construct
IINPRGDGTRYAQKFQG

SEQ ID NO: 83 - protein, 18 aa, synthetic construct
IINPRGDGTRYAQKFQGR

SEQ ID NO: 84 - protein, 19 aa, synthetic construct
IINPRGDGTRYAQKFQGRV

SEQ ID NO: 85 - protein, 20 aa, synthetic construct
IINPRGDGTRYAQKFQGRVT

SEQ ID NO: 86 - protein, 21 aa, synthetic construct
IINPRGDGTRYAQKFQGRVTM

SEQ ID NO: 87 - protein, 22 aa, synthetic construct
IINPRGDGTRYAQKFQGRVTMT

SEQ ID NO: 88 - protein, 7 aa, synthetic construct
INPRGDG

SEQ ID NO: 89 - protein, 8 aa, synthetic construct
INPRGDGT

SEQ ID NO: 90 - protein, 9 aa, synthetic construct
INPRGDGTR

SEQ ID NO: 91 - protein, 10 aa, synthetic construct
INPRGDGTRY

SEQ ID NO: 92 - protein, 11 aa, synthetic construct
INPRGDGTRYA

SEQ ID NO: 93 - protein, 12 aa, synthetic construct
INPRGDGTRYAQ

SEQ ID NO: 94 - protein, 13 aa, synthetic construct
INPRGDGTRYAQK

SEQ ID NO: 95 - protein, 14 aa, synthetic construct
INPRGDGTRYAQKF

SEQ ID NO: 96 - protein, 15 aa, synthetic construct
INPRGDGTRYAQKFQ

SEQ ID NO: 97 - protein, 16 aa, synthetic construct
INPRGDGTRYAQKFQG

SEQ ID NO: 98 - protein, 17 aa, synthetic construct
INPRGDGTRYAQKFQGR

SEQ ID NO: 99 - protein, 18 aa, synthetic construct
INPRGDGTRYAQKFQGRV

SEQ ID NO: 100 - protein, 19 aa, synthetic construct
INPRGDGTRYAQKFQGRVT

SEQ ID NO: 101 - protein, 20 aa, synthetic construct
INPRGDGTRYAQKFQGRVTM

SEQ ID NO: 102 - protein, 21 aa, synthetic construct
INPRGDGTRYAQKFQGRVTMT

SEQ ID NO: 103 - protein, 15 aa, synthetic construct
LEWMGIINPRGDGTR

SEQ ID NO: 104 - protein, 16 aa, synthetic construct
LEWMGIINPRGDGTRY

SEQ ID NO: 105 - protein, 17 aa, synthetic construct
LEWMGIINPRGDGTRYA

SEQ ID NO: 106 - protein, 18 aa, synthetic construct
LEWMGIINPRGDGTRYAQ

SEQ ID NO: 107 - protein, 19 aa, synthetic construct
LEWMGIINPRGDGTRYAQK

SEQ ID NO: 108 - protein, 20 aa, synthetic construct
LEWMGIINPRGDGTRYAQKF

SEQ ID NO: 109 - protein, 21 aa, synthetic construct
LEWMGIINPRGDGTRYAQKFQ

SEQ ID NO: 110 - protein, 22 aa, synthetic construct
LEWMGIINPRGDGTRYAQKFQG

SEQ ID NO: 111 - protein, 23 aa, synthetic construct
LEWMGIINPRGDGTRYAQKFQGR

SEQ ID NO: 112 - protein, 24 aa, synthetic construct
LEWMGIINPRGDGTRYAQKFQGRV

SEQ ID NO: 113 - protein, 25 aa, synthetic construct
LEWMGIINPRGDGTRYAQKFQGRVT

SEQ ID NO: 114 - protein, 26 aa, synthetic construct
LEWMGIINPRGDGTRYAQKFQGRVTM

SEQ ID NO: 115 - protein, 27 aa, synthetic construct
LEWMGIINPRGDGTRYAQKFQGRVTMT

SEQ ID NO: 116 - protein, 10 aa, synthetic construct
MGIINPRGDG

SEQ ID NO: 117 - protein, 11 aa, synthetic construct
MGIINPRGDGT

SEQ ID NO: 118 - protein, 12 aa, synthetic construct
MGIINPRGDGTR

SEQ ID NO: 119 - protein, 13 aa, synthetic construct
MGIINPRGDGTRY

SEQ ID NO: 120 - protein, 14 aa, synthetic construct
MGIINPRGDGTRYA

SEQ ID NO: 121 - protein, 15 aa, synthetic construct
MGIINPRGDGTRYAQ

SEQ ID NO: 122 - protein, 16 aa, synthetic construct
MGIINPRGDGTRYAQK

SEQ ID NO: 123 - protein, 17 aa, synthetic construct
MGIINPRGDGTRYAQKF

SEQ ID NO: 124 - protein, 18 aa, synthetic construct
MGIINPRGDGTRYAQKFQ

SEQ ID NO: 125 - protein, 19 aa, synthetic construct
MGIINPRGDGTRYAQKFQG

SEQ ID NO: 126 - protein, 20 aa, synthetic construct
MGIINPRGDGTRYAQKFQGR

SEQ ID NO: 127 - protein, 21 aa, synthetic construct
MGIINPRGDGTRYAQKFQGRV

SEQ ID NO: 128 - protein, 22 aa, synthetic construct
MGIINPRGDGTRYAQKFQGRVT

SEQ ID NO: 129 - protein, 23 aa, synthetic construct
MGIINPRGDGTRYAQKFQGRVTM

SEQ ID NO: 130 - protein, 24 aa, synthetic construct
MGIINPRGDGTRYAQKFQGRVTMT

SEQ ID NO: 131 - protein, 6 aa, synthetic construct
NPRGDG

SEQ ID NO: 132 - protein, 7 aa, synthetic construct
NPRGDGT

SEQ ID NO: 133 - protein, 8 aa, synthetic construct
NPRGDGTR

SEQ ID NO: 134 - protein, 9 aa, synthetic construct
NPRGDGTRY

SEQ ID NO: 135 - protein, 10 aa, synthetic construct
NPRGDGTRYA

SEQ ID NO: 136 - protein, 11 aa, synthetic construct
NPRGDGTRYAQ

SEQ ID NO: 137 - protein, 12 aa, synthetic construct
NPRGDGTRYAQK

SEQ ID NO: 138 - protein, 13 aa, synthetic construct
NPRGDGTRYAQKF

SEQ ID NO: 139 - protein, 14 aa, synthetic construct
NPRGDGTRYAQKFQ

SEQ ID NO: 140 - protein, 15 aa, synthetic construct
NPRGDGTRYAQKFQG

SEQ ID NO: 141 - protein, 16 aa, synthetic construct
NPRGDGTRYAQKFQGR

SEQ ID NO: 142 - protein, 17 aa, synthetic construct
NPRGDGTRYAQKFQGRV

SEQ ID NO: 143 - protein, 18 aa, synthetic construct
NPRGDGTRYAQKFQGRVT

SEQ ID NO: 144 - protein, 19 aa, synthetic construct
NPRGDGTRYAQKFQGRVTM

SEQ ID NO: 145 - protein, 20 aa, synthetic construct
NPRGDGTRYAQKFQGRVTMT

SEQ ID NO: 146 - protein, 10 aa, synthetic construct
PRGDGTRYAQ

SEQ ID NO: 147 - protein, 11 aa, synthetic construct
PRGDGTRYAQK

SEQ ID NO: 148 - protein, 12 aa, synthetic construct
PRGDGTRYAQKF

SEQ ID NO: 149 - protein, 13 aa, synthetic construct
PRGDGTRYAQKFQ

SEQ ID NO: 150 - protein, 14 aa, synthetic construct
PRGDGTRYAQKFQG

SEQ ID NO: 151 - protein, 15 aa, synthetic construct
PRGDGTRYAQKFQGR

SEQ ID NO: 152 - protein, 16 aa, synthetic construct
PRGDGTRYAQKFQGRV

SEQ ID NO: 153 - protein, 17 aa, synthetic construct
PRGDGTRYAQKFQGRVT

SEQ ID NO: 154 - protein, 18 aa, synthetic construct
PRGDGTRYAQKFQGRVTM

SEQ ID NO: 155 - protein, 19 aa, synthetic construct
PRGDGTRYAQKFQGRVTMT

SEQ ID NO: 156 - protein, 11 aa, synthetic construct
QGLEWMGIINP

SEQ ID NO: 157 - protein, 12 aa, synthetic construct
QGLEWMGIINPR

SEQ ID NO: 158 - protein, 13 aa, synthetic construct
QGLEWMGIINPRG

SEQ ID NO: 159 - protein, 14 aa, synthetic construct
QGLEWMGIINPRGD

SEQ ID NO: 160 - protein, 15 aa, synthetic construct
QGLEWMGIINPRGDG

SEQ ID NO: 161 - protein, 16 aa, synthetic construct
QGLEWMGIINPRGDGT

SEQ ID NO: 162 - protein, 17 aa, synthetic construct
QGLEWMGIINPRGDGTR

SEQ ID NO: 163 - protein, 18 aa, synthetic construct
QGLEWMGIINPRGDGTRY

SEQ ID NO: 164 - protein, 19 aa, synthetic construct
QGLEWMGIINPRGDGTRYA

SEQ ID NO: 165 - protein, 20 aa, synthetic construct
QGLEWMGIINPRGDGTRYAQ

SEQ ID NO: 166 - protein, 21 aa, synthetic construct
QGLEWMGIINPRGDGTRYAQK

SEQ ID NO: 167 - protein, 22 aa, synthetic construct
QGLEWMGIINPRGDGTRYAQKF

SEQ ID NO: 168 - protein, 8 aa, synthetic construct
RGDGTRYA

SEQ ID NO: 169 - protein, 9 aa, synthetic construct
RGDGTRYAQ

SEQ ID NO: 170 - protein, 10 aa, synthetic construct
RGDGTRYAQK

SEQ ID NO: 171 - protein, 11 aa, synthetic construct
RGDGTRYAQKF

SEQ ID NO: 172 - protein, 12 aa, synthetic construct
RGDGTRYAQKFQ

SEQ ID NO: 173 - protein, 13 aa, synthetic construct
RGDGTRYAQKFQG

SEQ ID NO: 174 - protein, 14 aa, synthetic construct
RGDGTRYAQKFQGR

SEQ ID NO: 175 - protein, 15 aa, synthetic construct
RGDGTRYAQKFQGRV

SEQ ID NO: 176 - protein, 16 aa, synthetic construct
RGDGTRYAQKFQGRVT

SEQ ID NO: 177 - protein, 17 aa, synthetic construct
RGDGTRYAQKFQGRVTM

SEQ ID NO: 178 - protein, 18 aa, synthetic construct
RGDGTRYAQKFQGRVTMT

SEQ ID NO: 179 - protein, 11 aa, synthetic construct
WMGIINPRGDG

SEQ ID NO: 180 - protein, 12 aa, synthetic construct
WMGIINPRGDGT

SEQ ID NO: 181 - protein, 13 aa, synthetic construct
WMGIINPRGDGTR

SEQ ID NO: 182 - protein, 14 aa, synthetic construct
WMGIINPRGDGTRY

SEQ ID NO: 183 - protein, 15 aa, synthetic construct
WMGIINPRGDGTRYA

SEQ ID NO: 184 - protein, 16 aa, synthetic construct
WMGIINPRGDGTRYAQ

SEQ ID NO: 185 - protein, 17 aa, synthetic construct
WMGIINPRGDGTRYAQK

SEQ ID NO: 186 - protein, 18 aa, synthetic construct
WMGIINPRGDGTRYAQKF

SEQ ID NO: 187 - protein, 19 aa, synthetic construct
WMGIINPRGDGTRYAQKFQ

SEQ ID NO: 188 - protein, 20 aa, synthetic construct
WMGIINPRGDGTRYAQKFQG

SEQ ID NO: 189 - protein, 21 aa, synthetic construct
WMGIINPRGDGTRYAQKFQGR

SEQ ID NO: 190 - protein, 22 aa, synthetic construct
WMGIINPRGDGTRYAQKFQGRV

SEQ ID NO: 191 - protein, 23 aa, synthetic construct
WMGIINPRGDGTRYAQKFQGRVT

SEQ ID NO: 192 - protein, 24 aa, synthetic construct
WMGIINPRGDGTRYAQKFQGRVTM

SEQ ID NO: 193 - protein, 25 aa, synthetic construct
WMGIINPRGDGTRYAQKFQGRVTMT

SEQ ID NO: 194 - protein, 5 aa, synthetic construct
YFYLH

SEQ ID NO: 195 - protein, 9 aa, synthetic construct
GADHGAFDI

SEQ ID NO: 196 - protein, 12 aa, synthetic construct
RASQSVRRNYFA

SEQ ID NO: 197 - protein, 7 aa, synthetic construct
DASTRAT

SEQ ID NO: 198 - protein, 11 aa, synthetic construct
QQYDSSPPMYI

(19) United States
(12) Patent Application Publication
(10) Pub. No.: US 2026/0193327 A1
(43) Pub. Date: Jul. 9, 2026

(54) SARS-CoV-2 Binding Antibody

(71) Applicant: Leyden Laboratories B.V., Leiden (NL)

(72) Inventor: Fergus Manford, Leiden (NL)

(21) Appl. No.: 19/127,632

(22) PCT Filed: Nov. 6, 2023
(86) PCT No.: PCT/NL2023/050584

§ 371 Date: May 6, 2025

(30) Foreign Application Priority Data
Nov. 6, 2022 (EP) 22205678.0

(51) Int. Cl. C07K 16/104; A61K 9/00; A61K 39/00; A61K 47/36; A61P 31/14

(52) U.S. Cl. CPC C07K 16/104; A61K 9/0053; A61K 47/36; A61P 31/14; A61K 2039/505; A61K 2039/543; A61K 2039/545; C07K 2317/14; C07K 2317/565

(57) ABSTRACT

The invention is in the field of medical treatment, and relates to a method for treating SARS-CoV-2 infections. In particular, the present invention relates to methods for prophylactic and/or therapeutic treatment of betacoronavirus infections, in particular, SARS-CoV-2 infections by means of intranasal administration or oral inhalation of antibodies.

### CROSS REFERENCE TO RELATED APPLICATIONS

This application is a national phase entry under 35 U.S.C. § 371 of International Patent Application PCT/NL2023/050584, filed Nov. 6, 2022, designating the United States of America and published in English as International Patent Publication WO 2024/096743 on May 10, 2024, which claims the benefit under Article 8 of the Patent Cooperation Treaty to European Patent Application Serial No. 22205678.0, filed Nov. 6, 2022, the entireties of which are hereby incorporated by reference.

### INCORPORATION BY REFERENCE

The ST.26 XML Sequence listing named “10909-10952-US-sequence listing”, created on Dec. 16, 2023, and having a size of 176,128 bytes, is hereby incorporated herein by this reference in its entirety.

### FIELD OF THE INVENTION

The invention is in the field of medical treatment and relates to a method for treating α- and β-coronavirus infections in animals and humans, including the Severe Acute Respiratory Syndrome Coronavirus 2 (SARS-CoV-2). In particular, the present invention relates to methods for prophylactic and/or therapeutic treatment of SARS-CoV-2 by means of intranasal administration and/or oral inhalation of antibodies against SARS-CoV-2.

### BACKGROUND OF THE INVENTION

The SARS-CoV-2 virus causes the disease Covid-19 in humans and is widespread having a significant impact on human society. We should expect the SARS-CoV-2 virus and its variants of concern to continue to cause severe disease and death. In recent years, other coronaviruses have been involved in several major pandemics, causing even more fatalities. As scientists develop therapeutic antibodies and vaccines against SARS-CoV-2, the danger remains of future novel coronaviruses emerging. These emerging and novel coronaviruses can lead to further pandemics. It remains vitally important to identify broadly protective therapies that can combat current and emerging coronaviruses in the future.

In addition to SARS-CoV-2, six other coronaviruses are known to cause human disease: the alphacoronaviruses HCoV-229E (human coronavirus 229E) and HCoV-NL63 (human coronavirus NL63), as well as the betacoronaviruses HCoV-OC43 (human coronavirus OC43), HCoV-HKU1 (human coronavirus HKU1), SARS-COV (severe acute respiratory syndrome coronavirus) and MERS-COV (Middle East respiratory syndrome coronavirus). Recently, three betacoronaviruses crossed from animals to humans, including SARS-CoV-2 causing serious outbreaks. As of October 2022, the ongoing COVID-19 pandemic caused by SARS-CoV-2, has resulted in more than 6.6 million worldwide deaths since 2019.

Furthermore, two coronaviruses previously linked only to animal infection were recently detected in humans who presented with flu-like symptoms.

Coronaviruses derive their name from their crown-like appearance. Coronaviruses are a large group of viruses that have spike proteins on their surface, resembling crown-like thorns.

The protection afforded by current COVID-19 vaccines arises from therapeutic monoclonal antibodies (mAbs) that target the receptor binding domain (RBD) present on these spike proteins.

### Structure of the Spike Protein

Coronavirus infection is a multistep process that involves enzymatic cleavage and rearrangement of the surface spike protein. The spike protein has an S1 subunit that binds host cell receptors and an S2 subunit that fuses the viral and cell membranes thereby facilitating cell entry.

The viral spike protein of SARS-CoV-2 facilitates viral entry by binding to the angiotensin-converting enzyme 2 (ACE2) receptor on human cells.

The SARS-CoV-2 spike contains two cleavage sites: a furin cleavage site at the boundary of the S1 and S2 subunits, and an S2′ site that is conserved in coronaviruses.

### S1 Subunit

The SARS-CoV-2 spike protein uses the RBD on the S1 subunit to engage the target cell's ACE2 receptor.

The S1 subunit is more accessible and remains the main target of many neutralizing antibodies. The S1 subunit, however, is more genetically variable than the S2 subunit. When subjected to the selective pressure from antibodies, this propensity towards genetic variability can lead to viral variants with the predominant changes occurring on the S1 subunit. Despite many variants, this broad array in variability is still able to effect receptor binding.

### S2 Subunit

The viral spike components essential to infection also involve the structurally complex S2 subunit. The S2 subunit contains dynamic elements essential for fusion with the host cell. Once the receptor has bound, the S1 subunit is discarded and the membrane enzyme transmembrane serine protease 2 (TMPRSS2) or endosomal cathepsins cleave the S2 site.

This cleaving leads to insertion of the fusion peptide into the cell membrane culminating in viral fusion.

It is understandable that given the very precise sequence of events required by the S2 element, these S2 subunit elements are less disposed towards genetic variability than the RBD, which so far has been capable of retaining or even increasing binding capabilities to ACE2 despite a variety of mutations.

The S2 domain sites yield poorly accessible targets for novel therapeutics to protect against a wider range of coronaviruses.

### The Helix

The stem helix at the base of the viral spike protein is even more difficult to access than elements on the S2 subunit but historically benefits from better-preserved amino acid sequencing.

### The Host's Defences

The antibodies that our body produces in response to an injected vaccine operates in the bloodstream and only comes into play once the virus starts to attack our circulatory system. The nasal vaccine interacts with a completely different part of our immune system. The nasal passages have their own defence mechanisms that are not triggered through an injection of the coronavirus vaccine, and do not interface well with antibodies in our blood. This means that when the coronavirus starts to colonize our nasal passages we have no defences.

### Treatments

The most common treatment method for individuals suffering from SARS-CoV-2 infections is symptomatic treatment. Limited options for the viral treatment of SARS-CoV-2 exist in the form of small molecule antiviral drugs. Thus, there is a need for different treatments, especially for treatments that are able to target a multitude of SARS-CoV-2 variants of concern at once such as broad-neutralizing antibodies (bnAbs).

Although treatment of SARS-CoV-2 infection with one, two or more antibodies is a possibility, a treatment comprising a single antibody or combination of antibodies would be considerably more cost-efficient.

It remains vitally important to identify broadly protective therapies that can combat novel coronaviruses. We have surprisingly developed a novel array of broadly protective antibodies which are disclosed herein.

### SUMMARY OF THE INVENTION

### Benefits

Unlike the prior art which disclose antibodies that bind to the S1 subunit, the interfering antibodies disclosed herein bind to and disrupt the functioning of the stem helix bundle thereby inhibiting membrane fusion of the SARS-CoV-2 virion with the target cell.

Furthermore, directing these interfering antibodies at the stem helix bundle allows for broader variant coverage than would otherwise be achieved when targeting the S1 subunit. Without wishing to be bound by theory, it is postulated that the interfering antibodies disclosed herein bind to the hydrophobic core of the stem helix bundle and disrupts its quaternary structure. The interfering antibodies disclosed herein bind to and disrupt the functioning of the stem helix bundle and inhibit membrane fusion. The interfering antibodies disclosed herein bind in a manner that likely prevents the S2 subunit refolding from the pre- to the post-fusion state and thereby blocks viral entry.

The interfering antibodies disclosed herein also allow for the addition of a broader array of formulation strategies to be used.

The use of interfering antibodies directed at the stem helix bundle have higher affinity for their targets than antibodies in the prior art, permitting lower doses as compared to antibodies to be used.

### SPECIFIC EMBODIMENTS

In a preferred embodiment, we disclose an improved composition comprising an antibody comprising a heavy chain variable domain that comprises a heavy chain CDR2 region comprising an amino acid sequence according to any one of SEQ ID NOS: 001 to 193 and a pharmaceutically acceptable excipient.

In a preferred embodiment, we disclose an improved series of antibodies comprising a heavy chain variable domain that comprises a heavy chain CDR2 region as described in any of SEQ ID NOS: 001 to 193.

A preferred embodiment comprises an antibody comprising a heavy chain variable domain that comprises a heavy chain CDR2 region comprising an amino acid sequence according to any one of SEQ ID NOS: 001 to 014.

A preferred embodiment comprises an antibody comprising a heavy chain variable domain that comprises a heavy chain CDR2 region comprising an amino acid sequence according to any one of SEQ ID NOS: 015 to 025.

A preferred embodiment comprises an antibody comprising a heavy chain variable domain that comprises a heavy chain CDR2 region comprising an amino acid sequence according to any one of SEQ ID NOS: 026 to 040.

A preferred embodiment comprises an antibody comprising a heavy chain variable domain that comprises a heavy chain CDR2 region comprising an amino acid sequence according to any one of SEQ ID NOS: 041 to 051.

A preferred embodiment comprises an antibody comprising a heavy chain variable domain that comprises a heavy chain CDR2 region comprising an amino acid sequence according to any one of SEQ ID NOS: 052 to 057.

A preferred embodiment comprises an antibody comprising a heavy chain variable domain that comprises a heavy chain CDR2 region comprising an amino acid sequence according to any one of SEQ ID NOS: 058 to 072. A preferred embodiment comprises an antibody comprising a heavy chain variable domain that comprises a heavy chain CDR2 region comprising an amino acid sequence according to any one of SEQ ID NOS: 073 to 087.

A preferred embodiment comprises an antibody comprising a heavy chain variable domain that comprises a heavy chain CDR2 region comprising an amino acid sequence according to any one of SEQ ID NOS: 088 to 102.

A preferred embodiment comprises an antibody comprising a heavy chain variable domain that comprises a heavy chain CDR2 region comprising an amino acid sequence according to any one of SEQ ID NOS: 103 to 115.

A preferred embodiment comprises an antibody comprising a heavy chain variable domain that comprises a heavy chain CDR2 region comprising an amino acid sequence according to any one of SEQ ID NOS: 116 to 130.

A preferred embodiment comprises an antibody comprising a heavy chain variable domain that comprises a heavy chain CDR2 region comprising an amino acid sequence according to any one of SEQ ID NOS: 131 to 145.

A preferred embodiment comprises an antibody comprising a heavy chain variable domain that comprises a heavy chain CDR2 region comprising an amino acid sequence according to any one of SEQ ID NOS: 146 to 155.

A preferred embodiment comprises an antibody comprising a heavy chain variable domain that comprises a heavy chain CDR2 region comprising an amino acid sequence according to any one of SEQ ID NOS: 156 to 167.

A preferred embodiment comprises an antibody comprising a heavy chain variable domain that comprises a heavy chain CDR2 region comprising an amino acid sequence according to any one of SEQ ID NOS: 168 to 178.

A preferred embodiment comprises an antibody comprising a heavy chain variable domain that comprises a heavy chain CDR2 region comprising an amino acid sequence according to any one of SEQ ID NOS: 179 to 193.

A preferred embodiment comprises an antibody comprising a heavy chain variable domain that comprises a heavy chain CDR2 region comprising an amino acid sequence according to any one of SEQ ID NOS: 009 to 014, SEQ ID NOS: 035 to 040, SEQ ID NOS: 048 to 051, SEQ ID NOS: 082 to 087, SEQ ID NOS: 110 to 115, SEQ ID NOS: 125 to 130 and SEQ ID NOS: 188 to 193.

A preferred embodiment comprises an antibody comprising a heavy chain variable domain that comprises a heavy chain CDR2 region comprising an amino acid sequence according to any one of SEQ ID NOS: 035, 036, 082, 083 or 0127.

A preferred embodiment comprises an antibody comprising a heavy chain variable domain that comprises a heavy chain CDR2 region comprising an amino acid sequence according to SEQ ID NO: 082.

A preferred embodiment comprises an antibody comprising a heavy chain variable domain that comprises a heavy chain CDR2 region consisting of an amino acid sequence according to any one of SEQ ID NOS: 001 to 014.

A preferred embodiment comprises an antibody comprising a heavy chain variable domain that comprises a heavy chain CDR2 region consisting of amino acid sequence according to any one of SEQ ID NOS: 015 to 025.

A preferred embodiment comprises an antibody comprising a heavy chain variable domain that comprises a heavy chain CDR2 region consisting of amino acid sequence according to any one of SEQ ID NOS: 026 to 040.

A preferred embodiment comprises an antibody comprising a heavy chain variable domain that comprises a heavy chain CDR2 region consisting of amino acid sequence according to any one of SEQ ID NOS: 041 to 051.

A preferred embodiment comprises an antibody comprising a heavy chain variable domain that comprises a heavy chain CDR2 region consisting of amino acid sequence according to any one of SEQ ID NOS: 052 to 057.

A preferred embodiment comprises an antibody comprising a heavy chain variable domain that comprises a heavy chain CDR2 region consisting of amino acid sequence according to any one of SEQ ID NOS: 058 to 072.

A preferred embodiment comprises an antibody comprising a heavy chain variable domain that comprises a heavy chain CDR2 region consisting of amino acid sequence according to any one of SEQ ID NOS: 073 to 087.

A preferred embodiment comprises an antibody comprising a heavy chain variable domain that comprises a heavy chain CDR2 region consisting of amino acid sequence according to any one of SEQ ID NOS: 088 to 102.

A preferred embodiment comprises an antibody comprising a heavy chain variable domain that comprises a heavy chain CDR2 region consisting of amino acid sequence according to any one of SEQ ID NOS: 103 to 115.

A preferred embodiment comprises an antibody comprising a heavy chain variable domain that comprises a heavy chain CDR2 region consisting of amino acid sequence according to any one of SEQ ID NOS: 116 to 130.

A preferred embodiment comprises an antibody comprising a heavy chain variable domain that comprises a heavy chain CDR2 region consisting of amino acid sequence according to any one of SEQ ID NOS: 131 to 145.

A preferred embodiment comprises an antibody comprising a heavy chain variable domain that comprises a heavy chain CDR2 region consisting of amino acid sequence according to any one of SEQ ID NOS: 146 to 155.

A preferred embodiment comprises an antibody comprising a heavy chain variable domain that comprises a heavy chain CDR2 region consisting of amino acid sequence according to any one of SEQ ID NOS: 156 to 167.

A preferred embodiment comprises an antibody comprising a heavy chain variable domain that comprises a heavy chain CDR2 region consisting of amino acid sequence according to any one of SEQ ID NOS: 168 to 178.

A preferred embodiment comprises an antibody comprising a heavy chain variable domain that comprises a heavy chain CDR2 region consisting of amino acid sequence according to any one of SEQ ID NOS: 179 to 193.

A preferred embodiment comprises an antibody comprising a heavy chain variable domain that comprises a heavy chain CDR2 region consisting of amino acid sequence according to any one of SEQ ID NOS: 009 to 014, SEQ ID NOS: 035 to 040, SEQ ID NOS: 048 to 051, SEQ ID NOS: 082 to 087, SEQ ID NOS: 110 to 115, SEQ ID NOS: 125 to 130 and SEQ ID NOS: 188 to 193.

A preferred embodiment comprises an antibody comprising a heavy chain variable domain that comprises a heavy chain CDR2 region consisting of amino acid sequence according to any one of SEQ ID NOS: 035, 036, 082, 083 or 0127.

A preferred embodiment comprises an antibody comprising a heavy chain variable domain that comprises a heavy chain CDR2 region consisting of amino acid sequence according to SEQ ID NO: 082.

A preferred embodiment comprises an antibody comprising a heavy chain variable domain that comprises a heavy chain CDR2 region as disclosed herein, wherein the amino acid sequence has at least 80%, 81%, 82%, 83%, 84%, 85%, 86%, 87%, 88%, 89%, 90%, 91%, 92%, 93%, 94%, 95%, 96%, 97%, 98%, or at least 99% sequence identity.

A preferred embodiment comprises an antibody as disclosed herein, for use in a method for treatment of a betacoronavirus infection, preferably a SARS-CoV-2 virus infection or variants of the SARS-CoV-2 virus in an individual, wherein the antibody is administered intranasally and/or by oral inhalation.

A preferred embodiment comprises an antibody as disclosed herein, wherein the method for treatment of SARS-CoV-2 virus infection is a method for prophylactic and/or therapeutic treatment of SARS-CoV-2 virus infection.

A preferred embodiment comprises an antibody as disclosed herein, wherein the antibody comprising a heavy chain variable domain that comprises a heavy chain CDR2 region that comprises at most any one of 1, 2 or 3 amino acid insertions, deletions or substitutions.

A preferred embodiment comprises an antibody as disclosed herein, wherein the antibody is administered intranasally.

A preferred embodiment comprises an antibody as disclosed herein, wherein between 0.01 μg and 20 mg of the antibody is administered to an individual.

A preferred embodiment comprises an antibody as disclosed herein, wherein the antibody is administered at least once or at least twice per day, preferably wherein the antibody is administered at least once or at least twice per week.

A preferred embodiment comprises a composition formulated for intranasal administration or oral inhalation comprising an antibody according to any other embodiment, in a single dose unit of between 0.01 μg and 20 mg.

A preferred embodiment comprises a composition wherein said antibody according to any other embodiment, is carried by a Lipid Nano-Particle (LNP), a liposome, or a virus-like particle (VLP).

A preferred embodiment comprises a nucleic acid molecule encoding an antibody according to any other embodiment disclosed herein.

A preferred embodiment comprises an expression vector comprising the nucleic acid molecule encoding an antibody according to any other embodiment disclosed herein.

A preferred embodiment comprises a host cell comprising the nucleic acid molecule encoding an antibody according to any other embodiment disclosed herein, or the expression vector comprising the nucleic acid molecule encoding an antibody according to any embodiment disclosed herein.

A preferred embodiment comprises a method of preparing an antibody, comprising: a) introducing an expression vector into the host cells; b) culturing the host cells in a culture medium, optionally comprising Isopropyl β-D-1-thiogalactopyranoside (IPTG) under conditions allowing the expression of the antibody in said host cells; c) lysing the host cells; d) separating the lysed cells, optionally into an insoluble fraction and a soluble fraction; e) solubilizing the lysed cells, optionally into an insoluble fraction using an extraction buffer, optionally comprising a chaotrophic agent and a reducing agent; and optionally f) dialyzing the solution obtained in step (e) in the presence of arginine at concentrations from 0.5M to 2M, preferably from 0.6M to 1.5M, preferably from 0.7M to 1M, thereby obtaining isolated antibodies.

A preferred embodiment comprises a composition comprising an antibody as disclosed herein for use in a method of prevention or treatment of a β-coronavirus infection, such as SARS-CoV-2 S protein.

A preferred embodiment comprises an antibody as disclosed herein that binds to an epitope in the viral envelope spike protein(S) of a β-coronavirus, such as SARS-CoV-2.

A preferred embodiment comprises an antibody as disclosed herein that binds to an epitope that is outside the RBD of a β-coronavirus, such as SARS-CoV-2 S protein.

A preferred embodiment comprises an antibody as disclosed herein that neutralizes a β-coronavirus, such as SARS-CoV-2.

A preferred embodiment comprises an antibody as disclosed herein that inhibits viral and cell membrane fusion.

A preferred embodiment comprises an antibody as disclosed herein that binds to the S1 subunit of a β-coronavirus, such as SARS-CoV-2 S protein.

A preferred embodiment comprises an antibody as disclosed herein that binds to the S2 subunit of a β-coronavirus, such as SARS-CoV-2 S protein.

A preferred embodiment comprises an antibody as disclosed herein that binds to the stem helix at the base of the viral spike protein of a β-coronavirus, such as SARS-CoV-2.

A preferred embodiment comprises an antibody as disclosed herein that also binds to the S protein of at least two β-coronaviruses, such as SARS-CoV-1 and SARS-CoV-2.

A preferred embodiment comprises an antibody as disclosed herein that also binds to the stem helix at the base of the viral spike protein of at least two β-coronaviruses, such as SARS-CoV-1 and SARS-CoV-2.

A preferred embodiment comprises an antibody as disclosed herein that exhibits broad 6-coronavirus neutralization by a stem helix-specific binding. In a preferred embodiment, the isolated antibodies disclosed herein, binds to α- and β-coronaviruses with a KD value of about 100 nM or lower, preferably about 10 nM or lower, preferably about 1 nM or lower, preferably about 100 pM or lower, preferably about 10 pM or lower, preferably about 1 pM or lower, preferably about 0.1 pM or lower.

In a preferred embodiment, the isolated antibodies disclosed herein, binds to an α-coronavirus with a KD value of about 100 nM or lower, preferably about 10 nM or lower, preferably about 1 nM or lower, preferably about 100 pM or lower, preferably about 10 pM or lower, preferably about 1 pM or lower, preferably about 0.1 pM or lower.

In a preferred embodiment, the isolated antibodies disclosed herein, binds to a β-coronavirus with a KD value of about 100 nM or lower, preferably about 10 nM or lower, preferably about 1 nM or lower, preferably about 100 pM or lower, preferably about 10 μM or lower, preferably about 1 μM or lower, preferably about 0.1 μM or lower.

In a preferred embodiment, the isolated antibodies disclosed herein, binds to SARS-CoV-2 with a KD value of about 100 nM or lower, preferably about 10 nM or lower, preferably about 1 nM or lower, preferably about 100 μM or lower, preferably about 10 μM or lower, preferably about 1 μM or lower, preferably about 0.1 μM or lower.

In a preferred embodiment, the isolated antibodies disclosed herein, binds to MERS-COV with a KD value of about 100 nM or lower, preferably about nM or lower, preferably about 1 nM or lower, preferably about 100 μM or lower, preferably about 10 μM or lower, preferably about 1 μM or lower, preferably about 0.1 μM or lower.

In a preferred embodiment, the antibodies as disclosed herein may be delivered to any mucosal environment. Specific mucosal environments include the nasal epithelium, the oropharyngeal epithelium, the digestive tract, the corneal epithelium or the pulmonary epithelium.

In a preferred embodiment, the antibodies as disclosed herein may be delivered to a combination of any of the epithelia mentioned immediately above.

In a preferred embodiment, the antibodies as disclosed herein may be delivered to the nasal cavity.

In a preferred embodiment, the antibodies as disclosed herein may be delivered to the pulmonary epithelium.

In a preferred embodiment, the antibodies as disclosed herein may be delivered to the corneal epithelium.

In a preferred embodiment, the antibodies as disclosed herein may be delivered to the nasal cavity, pulmonary epithelium and corneal epithelium.

The antibodies as disclosed herein are useful for the treatment of SARS-CoV-2 virus infection in an individual. Preferably, the method for treatment of SARS-CoV-2 virus infection is a method for prophylactic and/or therapeutic treatment of a SARS-CoV-2 virus infection. In some embodiments, an antibody is provided to an individual infected with SARS-CoV-2 virus. Preferably, the antibody is provided to the individual prophylactically. Preferably, the antibody is provided to the individual prior to SARS-CoV-2 virus infection.

In a preferred embodiment, the disclosure provides a method of treating SARS-CoV-2 virus infection in an individual, said method comprising administering intranasally or by oral inhalation to an individual in need thereof, an antibody comprising a heavy chain variable domain that comprises a heavy chain CDR2 region described in SEQ ID NOS: 001 to 193.

In some embodiments, the disclosure provides the antibody as disclosed herein for use in the manufacture of a medicament for use in treating SARS-CoV-2 virus infection.

Preferably, between 0.01 μg and 20 mg of the antibody is administered. Preferably, between 0.05 μg and 15 mg of the antibody is administered. Preferably, between 0.10 μg and 10 mg of the antibody is administered. Preferably, the antibody is administered at least once or at least twice daily. Preferably, the antibody is administered daily. Preferably, the antibody is administered at least once or at least twice weekly. Preferably, the antibody is administered at least once or at least twice monthly.

In a preferred embodiment, an antibody is disclosed comprising a heavy chain variable domain that comprises a heavy chain CDR1 region of SEQ ID NO: 194, a heavy chain CDR2 region of any SEQ ID NOS: 001 to 193, and a heavy chain CDR3 region of SEQ ID NO: 195 and a light chain CDR1 region of SEQ ID NO: 196, a light chain CDR2 region of SEQ ID NO: 197, and a light chain CDR3 region of SEQ ID NO: 198.

In a preferred embodiment, an antibody is disclosed comprising a heavy chain variable domain that comprises a heavy chain CDR1 region of SEQ ID NO: 194, a heavy chain CDR2 region of any SEQ ID NOS: 001 to 014, and a heavy chain CDR3 region of SEQ ID NO: 195 and a light chain CDR1 region of SEQ ID NO: 196, a light chain CDR2 region of SEQ ID NO: 197, and a light chain CDR3 region of SEQ ID NO: 198.

In a preferred embodiment, an antibody is disclosed comprising a heavy chain variable domain that comprises a heavy chain CDR1 region of SEQ ID NO: 194, a heavy chain CDR2 region of any SEQ ID NOS: 015 to 025, and a heavy chain CDR3 region of SEQ ID NO: 195 and a light chain CDR1 region of SEQ ID NO: 196, a light chain CDR2 region of SEQ ID NO: 197, and a light chain CDR3 region of SEQ ID NO: 198.

In a preferred embodiment, an antibody is disclosed comprising a heavy chain variable domain that comprises a heavy chain CDR1 region of SEQ ID NO: 194, a heavy chain CDR2 region of any SEQ ID NOS: 026 to 040, and a heavy chain CDR3 region of SEQ ID NO: 195 and a light chain CDR1 region of SEQ ID NO: 196, a light chain CDR2 region of SEQ ID NO: 197, and a light chain CDR3 region of SEQ ID NO: 198.

In a preferred embodiment, an antibody is disclosed comprising a heavy chain variable domain that comprises a heavy chain CDR1 region of SEQ ID NO: 194, a heavy chain CDR2 region of any SEQ ID NOS: 041 to 051, and a heavy chain CDR3 region of SEQ ID NO: 195 and a light chain CDR1 region of SEQ ID NO: 196, a light chain CDR2 region of SEQ ID NO: 197, and a light chain CDR3 region of SEQ ID NO: 198.

In a preferred embodiment, an antibody is disclosed comprising a heavy chain variable domain that comprises a heavy chain CDR1 region of SEQ ID NO: 194, a heavy chain CDR2 region of any SEQ ID NOS: 052 to 057, and a heavy chain CDR3 region of SEQ ID NO: 195 and a light chain CDR1 region of SEQ ID NO: 196, a light chain CDR2 region of SEQ ID NO: 197, and a light chain CDR3 region of SEQ ID NO: 198.

In a preferred embodiment, an antibody is disclosed comprising a heavy chain variable domain that comprises a heavy chain CDR1 region of SEQ ID NO: 194, a heavy chain CDR2 region of any SEQ ID NOS: 058 to 072, and a heavy chain CDR3 region of SEQ ID NO: 195 and a light chain CDR1 region of SEQ ID NO: 196, a light chain CDR2 region of SEQ ID NO: 197, and a light chain CDR3 region of SEQ ID NO: 198.

In a preferred embodiment, an antibody is disclosed comprising a heavy chain variable domain that comprises a heavy chain CDR1 region of SEQ ID NO: 194, a heavy chain CDR2 region of any SEQ ID NOS: 073 to 087, and a heavy chain CDR3 region of SEQ ID NO: 195 and a light chain CDR1 region of SEQ ID NO: 196, a light chain CDR2 region of SEQ ID NO: 197, and a light chain CDR3 region of SEQ ID NO: 198.

In a preferred embodiment, an antibody is disclosed comprising a heavy chain variable domain that comprises a heavy chain CDR1 region of SEQ ID NO: 194, a heavy chain CDR2 region of any SEQ ID NOS: 088 to 102, and a heavy chain CDR3 region of SEQ ID NO: 195 and a light chain CDR1 region of SEQ ID NO: 196, a light chain CDR2 region of SEQ ID NO: 197, and a light chain CDR3 region of SEQ ID NO: 198.

In a preferred embodiment, an antibody is disclosed comprising a heavy chain variable domain that comprises a heavy chain CDR1 region of SEQ ID NO: 194, a heavy chain CDR2 region of any SEQ ID NOS: 103 to 115, and a heavy chain CDR3 region of SEQ ID NO: 195 and a light chain CDR1 region of SEQ ID NO: 196, a light chain CDR2 region of SEQ ID NO: 197, and a light chain CDR3 region of SEQ ID NO: 198.

In a preferred embodiment, an antibody is disclosed comprising a heavy chain variable domain that comprises a heavy chain CDR1 region of SEQ ID NO: 194, a heavy chain CDR2 region of any SEQ ID NOS: 116 to 130, and a heavy chain CDR3 region of SEQ ID NO: 195 and a light chain CDR1 region of SEQ ID NO: 196, a light chain CDR2 region of SEQ ID NO: 197, and a light chain CDR3 region of SEQ ID NO: 198.

In a preferred embodiment, an antibody is disclosed comprising a heavy chain variable domain that comprises a heavy chain CDR1 region of SEQ ID NO: 194, a heavy chain CDR2 region of any SEQ ID NOS: 131 to 145, and a heavy chain CDR3 region of SEQ ID NO: 195 and a light chain CDR1 region of SEQ ID NO: 196, a light chain CDR2 region of SEQ ID NO: 197, and a light chain CDR3 region of SEQ ID NO: 198.

In a preferred embodiment, an antibody is disclosed comprising a heavy chain variable domain that comprises a heavy chain CDR1 region of SEQ ID NO: 194, a heavy chain CDR2 region of any SEQ ID NOS: 146 to 155, and a heavy chain CDR3 region of SEQ ID NO: 195 and a light chain CDR1 region of SEQ ID NO: 196, a light chain CDR2 region of SEQ ID NO: 197, and a light chain CDR3 region of SEQ ID NO: 198.

In a preferred embodiment, an antibody is disclosed comprising a heavy chain variable domain that comprises a heavy chain CDR1 region of SEQ ID NO: 194, a heavy chain CDR2 region of any SEQ ID NOS: 156 to 167, and a heavy chain CDR3 region of SEQ ID NO: 195 and a light chain CDR1 region of SEQ ID NO: 196, a light chain CDR2 region of SEQ ID NO: 197, and a light chain CDR3 region of SEQ ID NO: 198.

In a preferred embodiment, an antibody is disclosed comprising a heavy chain variable domain that comprises a heavy chain CDR1 region of SEQ ID NO: 194, a heavy chain CDR2 region of any SEQ ID NOS: 168 to 178, and a heavy chain CDR3 region of SEQ ID NO: 195 and a light chain CDR1 region of SEQ ID NO: 196, a light chain CDR2 region of SEQ ID NO: 197, and a light chain CDR3 region of SEQ ID NO: 198.

In a preferred embodiment, an antibody is disclosed comprising a heavy chain variable domain that comprises a heavy chain CDR1 region of SEQ ID NO: 194, a heavy chain CDR2 region of any SEQ ID NOS: 179 to 193, and a heavy chain CDR3 region of SEQ ID NO: 195 and a light chain CDR1 region of SEQ ID NO: 196, a light chain CDR2 region of SEQ ID NO: 197, and a light chain CDR3 region of SEQ ID NO: 198.

In a preferred embodiment, an antibody is disclosed comprising a heavy chain variable domain that comprises a heavy chain CDR1 region of SEQ ID NO: 194, a heavy chain CDR2 region of any SEQ ID NOS: 035, 036, 082, 083 or 0127, and a heavy chain CDR3 region of SEQ ID NO: 195 and a light chain CDR1 region of SEQ ID NO: 196, a light chain CDR2 region of SEQ ID NO: 197, and a light chain CDR3 region of SEQ ID NO: 198.

In a preferred embodiment, an antibody is disclosed comprising a heavy chain variable domain that comprises a heavy chain CDR1 region of SEQ ID NO: 194, a heavy chain CDR2 region of SEQ ID NO: 082, and a heavy chain CDR3 region of SEQ ID NO: 195 and a light chain CDR1 region of SEQ ID NO: 196, a light chain CDR2 region of SEQ ID NO: 197, and a light chain CDR3 region of SEQ ID NO: 198.

### DETAILED DESCRIPTION OF THE INVENTION

### Definitions

The term ‘prophylactic treatment’, as used herein, includes reference to a treatment for preventing infection of an individual with a SARS-CoV-2 virus. Prevention of an infection is preferably performed by administration of an antibody as disclosed herein prior to SARS-CoV-2 virus exposure. ‘Prophylactically’ therefore preferably means prior to virus exposure.

Nonetheless, it may involve limited invasion of the body by the virus, after which the virus does not, or not significantly, replicate. In a treatment method of the invention, prophylactic treatment involves administration of an antibody against SARS-CoV-2 virus at a point in time when the individual is not infected with a SARS-CoV-2 virus. Preferably, said antibody binds to the spike of a virion, more preferably a conserved epitope of the spike protein of a virion. In some embodiments, an individual in need thereof is not (yet) infected with SARS-CoV-2 virus.

The term ‘therapeutic treatment’, as used herein, includes reference to treatment of a viral infection (including SARS-CoV-2 disease) after viral infection has taken place. A viral infection involves the entry of the body by the virus, and the spreading of the virus to locations in the body other than the location of entry and/or the replication of the virus in the body. A viral infection may cause one or more disease, but may also be latent, in other words may reside in the body without causing a disease.

The term ‘SARS-CoV-2 virus infection’, as used herein, includes reference to the pathological or non-pathological, preferably pathological, entrance and residence of a SARS-CoV-2 virus of any type in a human host. The infecting virus may replicate within the host, its cells, or the cells of its microbiome. The infecting virus may or may not cause a disease, such as Covid-19. The infection may or may not be able to be detected by methods for virus infection detection known in the art. The infected individual may or may not be aware of the infection. Typical, but non-exclusive locations of the human body where SARS-CoV-2 may be located in an infected individual, are the respiratory system including the nasal passages and/or cells thereof, the ocular system and/or cells thereof, and the cardiovascular system and/or cells thereof. The term ‘SARS-CoV-2 virus infection’, as used herein, further includes reference to the entrance and residence of a part of a SARS-CoV-2 virus of any type that is able to cause viral replication in a human host. The term ‘SARS-CoV-2 virus infection’ encompasses SARS-CoV-2 disease (Severe Acute Respiratory Syndrome) and is preferably SARS-CoV-2 disease (Covid-19). The term ‘SARS-CoV-2 virus: infection’ can be used interchangeably with ‘SARS-CoV-2 viral infection’.

The term ‘individual’, as used herein, includes reference to a mammal or human that is subject to, or a risk of suffering from, SARS-CoV-2 viral infection. Infection may take place in any system, tissue or cell belonging to the host, including the host's microbiome. SARS-CoV-2 virus infection and the disease Covid-19 may occur in individuals of all age groups and sexes. Nonetheless, preferably, the individual is a human, in particular an elderly human such as a human that is at least 60, 65, 70, 75, 80, or at least 85 years old. Preferably, the individual is at risk of suffering from Severe Acute Respiratory Syndrome once infected. In embodiments, the individual has an underlying disease such as (i) a respiratory disease such as asthma, COPD, chronic bronchitis and lung emphysema, (ii) cardiovascular disease such as cardiac arrhythmia or individuals that have received cardiac surgery, (iii) diabetes, (iv) renal failure and/or (v) a disease affecting the immune system, for instance immunocompromised individuals.

The terms ‘administering’ and ‘administration’, as used herein, include reference to the provision of one or more drug and optionally one or more adjuvant with the aim to treat, cure, reduce, or prevent a disease or its symptoms in an individual, or to promote the individual's well-being. Preferred methods of administration of the antibody as disclosed herein include intranasal administration and oral inhalation.

The phrase ‘an individual in need thereof’, as used herein, includes reference to a mammal such as a human that benefits from a specified therapy. As a treatment method of the invention the antibodies as disclosed herein may be used prophylactically, the exhibition of symptoms or indications for SARS-CoV-2 virus infection are not required. Individuals that are especially in need of the method or antibody for use of the invention, are individuals with an elevated risk of SARS-CoV-2 virus infection, individuals with an elevated risk of Covid-19, individuals with an elevated risk of developing severe symptoms (illness) of Covid-19, and/or individuals with an elevated risk of dying from Covid-19. The person skilled in the art is aware of the risk factors for an elevated risk of SARS-CoV-2 virus infection, an elevated risk of Covid-19, an elevated risk of developing severe symptoms of Covid-19, and an elevated risk of dying from Covid-19.

The term ‘intranasally’, as used herein, may also be referred to as ‘nasal administration’, and includes reference to a route of administration in which a drug is provided into the upper respiratory tract, preferably through the nostrils, as part of a prophylactic and/or therapeutic treatment as disclosed herein. Preferably, the administration provides for drug in the nasal cavity. The back section of the nasal cavity is also referred to as the pharynx. Nasal administration preferably provides for delivery of antibodies as disclosed herein in the mucous membrane lining the nasal cavity. Intranasal administration can be performed by the use of for instance a nasal spray or nose drops. In some embodiments, the drug is delivered to the nasal cavity via the oral route. For example, RetroNose uses a breath-actuated pressurised metered-dose inhaler (pMDI) to administer drugs through the buccal cavity during the nasal expiratory phase. Such methods allow the drug particles to enter the nasal cavities through the rhinopharynx. Preferably, the antibodies of the invention are administered intranasally.

The term ‘oral inhalation’, as used herein, may also be referred to as ‘mouth inhalation’, and includes reference to a route of administration in which a drug is provided through the mouth to the lower respiratory tract such as the lungs, as part of a prophylactic and/or therapeutic treatment of the invention. Oral inhalation may for example be applied for drugs in their powdered form and drugs in the form of liquid droplets or aerosols.

Preferably, the antibodies of the invention are administered by oral inhalation.

The term ‘prior to’, as used herein, includes reference to the administration of an antibody before an individual has been exposed to, or is infected with, a SARS-CoV-2 virus. Preferably, the antibody as disclosed is administered to an individual up to 24 hours prior to SARS-CoV-2 virus exposure, for example between zero and 24 hours before the individual has been exposed to said SARS-CoV-2 virus. In some embodiments, the antibody is administered at least 2 days, 3 days, 4 days or more prior to SARS-CoV-2 virus exposure.

The term ‘dosage’ as used herein, refers to the amount of antibody to be given at a particular time (e.g., over the course of a 24-hour, 12-hour, 30 minute period, etc.). A dose refers to a single dosing episode, whether the dose is a unit dosage form or multiple unit dosage forms taken together (e.g., ingestion of two or more pills, receiving two or more injections). A dosage includes reference to a pharmaceutical dosage form wherein the medicament is packaged for administration as, e.g., a single-unit dose or multiple-unit dose. A dosage may also be administered as, e.g., one or more drops of an antibody-comprising composition (e.g., nasal drops or eye drops) or one or more sprays of an antibody-comprising composition (e.g., nasal sprays). Preferably, a suitable dosage of an antibody as disclosed herein contains a dose of between 0.01 μg and 20 mg, preferably 0.05 μg to 10 mg, more preferably around 0.10 μg to 5 mg or even around 1-2 mg, e.g. when the dosage is for intranasal administration. Such dosages are also referred to as “flat dosages” or “nominal doses” in contrast to dosages based on the weight of the patient. Flat dosages have the advantage that the medicament can be packaged in a single-unit dose, for example. A single dose of the antibody can provide protection from SARS-CoV-2 infection for several days and may be provided “on demand” or “as needed”. For example, an individual may administer the antibody before leaving the house or before coming into contact with other individuals.

In order to provide long-lasting protection, the antibody may be administered on a regular basis. For example, the antibody is administered once, or at least once per month. In a preferred embodiment, the antibody is administered once, or at least once per week, e.g., twice weekly. In a preferred embodiment, the antibody is administered once, or at least once per day. As is clear to a skilled person, less antibody may be administered when the antibody is administered more frequently (e.g., daily). In a preferred embodiment, between 0.1 mg to 20 mg of antibody is administered per week (e.g., once or twice weekly or daily). In an exemplary embodiment, 0.1 mg to 1 mg is administered daily (i.e, 0.7 mg to 7 mg per week). In an exemplary embodiment, 0.5 mg to 3.5 mg is administered twice weekly (i.e., 1 mg to 7 mg per week).

The present invention relates inter alia to an antibody for use in a method for treatment of SARS-CoV-2 virus infection in an individual, more specifically the antibody can be used in a method for the prophylactic and/or therapeutic treatment of SARS-CoV-2 virus infection in an individual.

Preferably, the individual is a mammal, more preferably a human. The route of administration of the antibody as disclosed herein is intranasally or by oral inhalation.

Preferably, an antibody as disclosed herein is capable of neutralizing at least one or more, preferably two or more, preferably three or more, preferably four or more, even more preferably five or more SARS-CoV-2 variants.

The antibody as disclosed herein may be capable of specifically binding to the S2 subunit of the spike protein of the SARS-CoV-2 virus.

Preferably, the antibody as disclosed herein is capable of specifically binding to an epitope in the stem region of the spike protein of the SARS-CoV-2 virus, more preferably, the antibody as disclosed herein binds to an epitope that is accessible in the pre-fusion conformation of the spike protein of the SARS-CoV-2 virus.

The antibody as disclosed herein may be capable of specifically binding to SARS-CoV-2 viruses that are in attenuated or inactivated form or that are viable, living and/or in the infective form.

Methods for attenuating or inactivating virus, e.g. SARS-CoV-2 viruses are well known in the art and include, but are not limited to, treatment with formalin, P-propiolactone (BPL), merthiolate, and/or ultraviolet light.

The antibody as disclosed herein may also be capable of specifically binding to one or more fragments of the SARS-CoV-2 viruse, such as one or more recombinantly produced proteins and/or antibodies of SARS-CoV-2 or a preparation of one or more proteins and/or (poly) peptides derived from variants of SARS-CoV-2.

The amino acid and/or nucleotide sequence of proteins of various SARS-CoV-2 variants can be found in the EMBL-database, GenBank-database, SARS-CoV-2 Sequence Database (ISD), and/or other databases. It is well within the reach of the skilled person to find such sequences in the respective databases.

An “epitope”, as used herein, includes reference to a moiety that is capable of binding to an antibody as disclosed herein with sufficiently high affinity to form a detectable antigen-antibody complex.

The antibody as disclosed herein can be used in isolated or non-isolated form.

The antibody as disclosed herein can be used alone or in a mixture comprising the antibody (or variant or fragment thereof) as disclosed herein. Furthermore, the antibodies as disclosed herein can be used with other antibodies that bind to SARS-CoV-2 and have SARS-CoV-2 virus inhibiting effect.

In other words, the antibody as disclosed herein can be used in combination, e.g., as a pharmaceutical composition comprising two or more antibodies that specifically bind SARS-CoV-2 virus. For example, antibodies having different, but complementary activities can be combined in a single therapy to achieve a desired therapeutic or prophylactic effect, but alternatively, antibodies having identical activities can also be combined in a single therapy to achieve a desired prophylactic or therapeutic effect. Optionally, the mixture further comprises at least one other therapeutic agent. Preferably, the therapeutic agent such as, e.g., neuraminidase inhibitors (e.g., zanamivir, oseltamivir) and/or M2 inhibitors (e.g., amantidine, rimantadine) is useful in the prophylaxis and/or treatment of a SARS-CoV-2 virus infection. Preferably, the composition comprises a single anti-SARS-CoV-2 virus, wherein the antibody is as disclosed herein.

The antibody as disclosed herein is preferably able to recognize and bind to the HA protein of SARS-CoV-2. Preferably, the antibody as disclosed herein is able to cross-neutralize SARS-CoV-2 variants. The skilled person can determine whether an antibody indeed cross-reacts with HA proteins from different variants of the SARS-CoV-2 virus.

An antibody as disclosed herein is administered intranasally or by oral inhalation.

With intranasal administration, an antibody is provided through the nostrils to the upper respiratory tract as part of a prophylactic and/or therapeutic treatment as disclosed herein. Preferably, the administration provides for antibody in the nasal cavity. The back section of the nasal cavity is also referred to as pharynx. Nasal administration can either be a form of topical administration or systemic administration, as the antibodies thus locally delivered can go on to have either local or systemic effects. In the present case, with antibodies as disclosed herein, nasal administration is preferably a form of topical administration. Nasal administration preferably provides for delivery of antibodies as disclosed herein in the mucous membrane lining the nasal cavity. Intranasal administration can be performed by the use of for instance a nasal spray or nose drops. When the composition is a nasal spray, the liquid aerosol will preferably comprise droplets smaller than 500 μm, preferably less than 100 μm in diameter (as measured using laser diffraction). These droplets will deposit the nasal passages and will therefore have a better deposition pattern.

Intranasal administration as disclosed herein may be done using a medicament in liquid form, preferably in the form of drops or nasal spray. The aqueous liquid may comprise adjuvants. These adjuvants may for example be salts, oils, cytokines, emulsifiers, buffering agents, carbohydrates and combinations thereof. Intranasal administration may also be done using a medicament in solid form, such as powders.

With oral inhalation, the antibody is provided through the mouth to the respiratory tract, preferably lower respiratory tract such as lungs, as part of a prophylactic and/or therapeutic treatment of the invention. As described further herein, oral inhalation also includes nasal drug delivery (also referred to as nasal drug delivery via the oral route). Oral inhalation may for example be applied for antibodies in their powdered form and antibodies in the form of liquid droplets or aerosols. Oral inhalation may include the use of an inhaler. The inhaler may be involved in the achievement of the dose that was determined. The antibody that is administered by oral inhalation may reach the lung, but may also partially be cleared out by exhalation.

Administration by oral inhalation as disclosed herein may be done using a medicament comprising aerosols in powdered (solid) or liquid form. Powdered aerosols comprising particles smaller than 5 μm in diameter will primarily reach the respiratory region of the lung, and will therefore be absorbed better than larger particles. The medicament may comprise adjuvants. These adjuvants may for example be salts, oils, cytokines, emulsifiers, buffering agents, carbohydrates and combinations thereof.

The compositions disclosed herein comprise pharmaceutically acceptable excipients. These pharmaceutically acceptable excipients may for example be salts, oils, cytokines, emulsifiers, buffering agents, carbohydrates and combinations thereof. Preferred pharmaceutically acceptable excipients include mucoadhesive excipients, for example mucoadhesive polymers, cellulose derivatives such as hypromellose (HPMC). Preferred mucoadhesive polymers have numerous hydrophilic groups, such as hydroxyl, carboxyl, amide, and sulfate. Preferred mucoadhesive polymers are thiolated polymers because these have improved tensile strength, rapid swelling, and advantageous water uptake behavior.

Preferred pharmaceutically acceptable excipients include mucoadhesive excipients. A preferred pharmaceutically acceptable excipient to be used with the composition according to the present invention is any one of chitosan, PVA, PVP, starch, pectin, poloxamer, carbopol, PVA, CMC, HPMC, PMVE and MA, or combination or two or three mucoadhesive excipients listed above.

Functionalized mucoadhesive polymers, such as polycarbophil, hyaluronan, and amberlite resin are also considered preferred pharmaceutically acceptable excipients.

Preferred pharmaceutically acceptable excipients also include gel forming agents.

Preferred pharmaceutically acceptable excipients also include film forming agents.

The invention also provides a composition formulated for intranasal administration and/or oral inhalation comprising an antibody as disclosed herein, preferably in a single dose unit between 0.01 μg to 20 mg or preferably 0.10 μg to 10 mg, wherein the antibody is as defined herein above in relation to the treatment methods.

In some embodiments, the antibodies as disclosed herein are the sole active ingredient in the treatment, e.g., the antibody is provided in a composition as the sole active ingredient. In some embodiments, the antibody disclosed herein is the sole antibody, in particular the sole anti-SARS-CoV-2 antibody, in the treatment, e.g., the antibody is provided in a composition as the sole antibody, in particular the sole anti-SARS-CoV-2 antibody. Preferably, compositions are providing comprising an anti-SARS-CoV-2 antibody consisting of the antibody disclosed herein. In such compositions, no other anti-SARS-CoV-2 antibodies are present.

Preferably, in a composition of the invention, the antibody is an antibody as disclosed in relation to a treatment method of the invention.

Preferably, the composition of the invention is a water-based composition such as an aqueous liquid. Preferably, the composition of the invention further comprises one or more salts, for example sodium chloride. The composition of the invention may further comprise one or more buffering agents, for example sodium acetate. The composition of the invention may further comprise one or more carbohydrates, such as sucrose, or other active ingredients, such as other antibodies, neuraminidase inhibitors, endonuclease inhibitors, and adjuvants such as oils, cytokines, emulsifiers, or combinations thereof. The pH of the composition of the invention can have a pH of between 4 and 7, more preferably around 5.5.

For the purpose of clarity and a concise description, features may be described herein as part of the same or separate embodiments, however, it will be appreciated that the disclosure includes embodiments having combinations of all or some of the features described. References to “method for treatment”, “treatment method”, “antibody for use” and “use of an antibody in the manufacture of a medicament” can be used interchangeably and embodiments disclosed in relation to any one of those aspects also applies in relation to said other aspects. In other words, they all refer to medical treatments involving an antibody as disclosed herein.

### Lipid Nano-Particle (LNP)

Preferably the antibodies disclosed herein are incorporated into a LNP. A LNP usually only have a single phospholipid outer layer that encapsulates the interior comprising the antibodies disclosed herein, the interior may be non-aqueous. By encapsulating the antibodies disclosed herein antibodies, a LNP protects the antibodies from proteases and thus ensures longer duration of activity of the antibodies.

### Sequences

CDRs and framework regions defined based on Kabat numbering:

Heavy chain CDR2 region
(SEQ ID NO: 001)
EWMGIINPRGDGT
Heavy chain CDR2 region
(SEQ ID NO: 002)
EWMGIINPRGDGTR
Heavy chain CDR2 region
(SEQ ID NO: 003)
EWMGIINPRGDGTRY
Heavy chain CDR2 region
(SEQ ID NO: 004)
EWMGIINPRGDGTRYA
Heavy chain CDR2 region
(SEQ ID NO: 005)
EWMGIINPRGDGTRYAQ
Heavy chain CDR2 region
(SEQ ID NO: 006)
EWMGIINPRGDGTRYAQK
Heavy chain CDR2 region
(SEQ ID NO: 007)
EWMGIINPRGDGTRYAQKF
Heavy chain CDR2 region
(SEQ ID NO: 008)
EWMGIINPRGDGTRYAQKFQ
Heavy chain CDR2 region
(SEQ ID NO: 009)
EWMGIINPRGDGTRYAQKFQG
Heavy chain CDR2 region
(SEQ ID NO: 010)
EWMGIINPRGDGTRYAQKFQGR
Heavy chain CDR2 region
(SEQ ID NO: 011)
EWMGIINPRGDGTRYAQKFQGRV
Heavy chain CDR2 region
(SEQ ID NO: 012)
EWMGIINPRGDGTRYAQKFQGRVT
Heavy chain CDR2 region
(SEQ ID NO: 013)
EWMGIINPRGDGTRYAQKFQGRVTM
Heavy chain CDR2 region
(SEQ ID NO: 014)
EWMGIINPRGDGTRYAQKFQGRVTMT
Heavy chain CDR2 region
(SEQ ID NO: 015)
GDGTRYA
Heavy chain CDR2 region
(SEQ ID NO: 016)
GDGTRYAQ
Heavy chain CDR2 region
(SEQ ID NO: 017)
GDGTRYAQK
Heavy chain CDR2 region
(SEQ ID NO: 018)
GDGTRYAQKF
Heavy chain CDR2 region
(SEQ ID NO: 019)
GDGTRYAQKFQ
Heavy chain CDR2 region
(SEQ ID NO: 020)
GDGTRYAQKFQG
Heavy chain CDR2 region
(SEQ ID NO: 021)
GDGTRYAQKFQGR
Heavy chain CDR2 region
(SEQ ID NO: 022)
GDGTRYAQKFQGRV
Heavy chain CDR2 region
(SEQ ID NO: 023)
GDGTRYAQKFQGRVT
Heavy chain CDR2 region
(SEQ ID NO: 024)
GDGTRYAQKFQGRVTM
Heavy chain CDR2 region
(SEQ ID NO: 025)
GDGTRYAQKFQGRVTMT
Heavy chain CDR2 region
(SEQ ID NO: 026)
GIINPRGDG
Heavy chain CDR2 region
(SEQ ID NO: 027)
GIINPRGDGT
Heavy chain CDR2 region
(SEQ ID NO: 028)
GIINPRGDGTR
Heavy chain CDR2 region
(SEQ ID NO: 029)
GIINPRGDGTRY
Heavy chain CDR2 region
(SEQ ID NO: 030)
GIINPRGDGTRYA
Heavy chain CDR2 region
(SEQ ID NO: 031)
GIINPRGDGTRYAQ
Heavy chain CDR2 region
(SEQ ID NO: 032)
GIINPRGDGTRYAQK
Heavy chain CDR2 region
(SEQ ID NO: 033)
GIINPRGDGTRYAQKF
Heavy chain CDR2 region
(SEQ ID NO: 034)
GIINPRGDGTRYAQKFQ
Heavy chain CDR2 region
(SEQ ID NO: 035)
GIINPRGDGTRYAQKFQG
Heavy chain CDR2 region
(SEQ ID NO: 036)
GIINPRGDGTRYAQKFQGR
Heavy chain CDR2 region
(SEQ ID NO: 037)
GIINPRGDGTRYAQKFQGRV
Heavy chain CDR2 region
(SEQ ID NO: 038)
GIINPRGDGTRYAQKFQGRVT
Heavy chain CDR2 region
(SEQ ID NO: 039)
GIINPRGDGTRYAQKFQGRVTM
Heavy chain CDR2 region
(SEQ ID NO: 040)
GIINPRGDGTRYAQKFQGRVTMT
Heavy chain CDR2 region
(SEQ ID NO: 041)
GLEWMGIINPRGDGTR
Heavy chain CDR2 region
(SEQ ID NO: 042)
GLEWMGIINPRGDGTRY
Heavy chain CDR2 region
(SEQ ID NO: 043)
GLEWMGIINPRGDGTRYA
Heavy chain CDR2 region
(SEQ ID NO: 044)
GLEWMGIINPRGDGTRYAQ
Heavy chain CDR2 region
(SEQ ID NO: 045)
GLEWMGIINPRGDGTRYAQK
Heavy chain CDR2 region
(SEQ ID NO: 046)
GLEWMGIINPRGDGTRYAQKF
Heavy chain CDR2 region
(SEQ ID NO: 047)
GLEWMGIINPRGDGTRYAQKFQ
Heavy chain CDR2 region
(SEQ ID NO: 048)
GLEWMGIINPRGDGTRYAQKFQG
Heavy chain CDR2 region
(SEQ ID NO: 049)
GLEWMGIINPRGDGTRYAQKFQGR
Heavy chain CDR2 region
(SEQ ID NO: 050)
GLEWMGIINPRGDGTRYAQKFQGRV
Heavy chain CDR2 region
(SEQ ID NO: 051)
GLEWMGIINPRGDGTRYAQKFQGRVT
Heavy chain CDR2 region
(SEQ ID NO: 052)
GQGLEWMGIINPRGDGTR
Heavy chain CDR2 region
(SEQ ID NO: 053)
GQGLEWMGIINPRGDGTRY
Heavy chain CDR2 region
(SEQ ID NO: 054)
GQGLEWMGIINPRGDGTRYA
Heavy chain CDR2 region
(SEQ ID NO: 055)
GQGLEWMGIINPRGDGTRYAQ
Heavy chain CDR2 region
(SEQ ID NO: 056)
GQGLEWMGIINPRGDGTRYAQK
Heavy chain CDR2 region
(SEQ ID NO: 057)
GQGLEWMGIINPRGDGTRYAQKF
Heavy chain CDR2 region
(SEQ ID NO: 058)
INPRGDG
Heavy chain CDR2 region
(SEQ ID NO: 059)
INPRGDGT
Heavy chain CDR2 region
(SEQ ID NO: 060)
INPRGDGTR
Heavy chain CDR2 region
(SEQ ID NO: 061)
INPRGDGTRY
Heavy chain CDR2 region
(SEQ ID NO: 062)
INPRGDGTRYA
Heavy chain CDR2 region
(SEQ ID NO: 063)
INPRGDGTRYAQ
Heavy chain CDR2 region
(SEQ ID NO: 064)
INPRGDGTRYAQK
Heavy chain CDR2 region
(SEQ ID NO: 065)
INPRGDGTRYAQKF
Heavy chain CDR2 region
(SEQ ID NO: 066)
INPRGDGTRYAQKFQ
Heavy chain CDR2 region
(SEQ ID NO: 067)
INPRGDGTRYAQKFQG
Heavy chain CDR2 region
(SEQ ID NO: 068)
INPRGDGTRYAQKFQGR
Heavy chain CDR2 region
(SEQ ID NO: 069)
INPRGDGTRYAQKFQGRV
Heavy chain CDR2 region
(SEQ ID NO: 070)
INPRGDGTRYAQKFQGRVT
Heavy chain CDR2 region
(SEQ ID NO: 071)
INPRGDGTRYAQKFQGRVTM
Heavy chain CDR2 region
(SEQ ID NO: 072)
INPRGDGTRYAQKFQGRVTMT
Heavy chain CDR2 region
(SEQ ID NO: 073)
IINPRGDG
Heavy chain CDR2 region
(SEQ ID NO: 074)
IINPRGDGT
Heavy chain CDR2 region
(SEQ ID NO: 075)
IINPRGDGTR
Heavy chain CDR2 region
(SEQ ID NO: 076)
IINPRGDGTRY
Heavy chain CDR2 region
(SEQ ID NO: 077)
IINPRGDGTRYA
Heavy chain CDR2 region
(SEQ ID NO: 078)
IINPRGDGTRYAQ
Heavy chain CDR2 region
(SEQ ID NO: 079)
IINPRGDGTRYAQK
Heavy chain CDR2 region
(SEQ ID NO: 080)
IINPRGDGTRYAQKF
Heavy chain CDR2 region
(SEQ ID NO: 081)
IINPRGDGTRYAQKFQ
Heavy chain CDR2 region
(SEQ ID NO: 082)
IINPRGDGTRYAQKFQG
Heavy chain CDR2 region
(SEQ ID NO: 083)
IINPRGDGTRYAQKFQGR
Heavy chain CDR2 region
(SEQ ID NO: 084)
IINPRGDGTRYAQKFQGRV
Heavy chain CDR2 region
(SEQ ID NO: 085)
IINPRGDGTRYAQKFQGRVT
Heavy chain CDR2 region
(SEQ ID NO: 086)
IINPRGDGTRYAQKFQGRVTM
Heavy chain CDR2 region
(SEQ ID NO: 087)
IINPRGDGTRYAQKFQGRVTMT
Heavy chain CDR2 region
(SEQ ID NO: 088)
INPRGDG
Heavy chain CDR2 region
(SEQ ID NO: 089)
INPRGDGT
Heavy chain CDR2 region
(SEQ ID NO: 090)
INPRGDGTR
Heavy chain CDR2 region
(SEQ ID NO: 091)
INPRGDGTRY
Heavy chain CDR2 region
(SEQ ID NO: 092)
INPRGDGTRYA
Heavy chain CDR2 region
(SEQ ID NO: 093)
INPRGDGTRYAQ
Heavy chain CDR2 region
(SEQ ID NO: 094)
INPRGDGTRYAQK
Heavy chain CDR2 region
(SEQ ID NO: 095)
INPRGDGTRYAQKF
Heavy chain CDR2 region
(SEQ ID NO: 096)
INPRGDGTRYAQKFQ
Heavy chain CDR2 region
(SEQ ID NO: 097)
INPRGDGTRYAQKFQG
Heavy chain CDR2 region
(SEQ ID NO: 098)
INPRGDGTRYAQKFQGR
Heavy chain CDR2 region
(SEQ ID NO: 099)
INPRGDGTRYAQKFQGRV
Heavy chain CDR2 region
(SEQ ID NO: 100)
INPRGDGTRYAQKFQGRVT
Heavy chain CDR2 region
(SEQ ID NO: 101)
INPRGDGTRYAQKFQGRVTM
Heavy chain CDR2 region
(SEQ ID NO: 102)
INPRGDGTRYAQKFQGRVTMT
Heavy chain CDR2 region
(SEQ ID NO: 103)
LEWMGIINPRGDGTR
Heavy chain CDR2 region
(SEQ ID NO: 104)
LEWMGIINPRGDGTRY
Heavy chain CDR2 region
(SEQ ID NO: 105)
LEWMGIINPRGDGTRYA
Heavy chain CDR2 region
(SEQ ID NO: 106)
LEWMGIINPRGDGTRYAQ
Heavy chain CDR2 region
(SEQ ID NO: 107)
LEWMGIINPRGDGTRYAQK
Heavy chain CDR2 region
(SEQ ID NO: 108)
LEWMGIINPRGDGTRYAQKF
Heavy chain CDR2 region
(SEQ ID NO: 109)
LEWMGIINPRGDGTRYAQKFQ
Heavy chain CDR2 region
(SEQ ID NO: 110)
LEWMGIINPRGDGTRYAQKFQG
Heavy chain CDR2 region
(SEQ ID NO: 111)
LEWMGIINPRGDGTRYAQKFQGR
Heavy chain CDR2 region
(SEQ ID NO: 112)
LEWMGIINPRGDGTRYAQKFQGRV
Heavy chain CDR2 region
(SEQ ID NO: 113)
LEWMGIINPRGDGTRYAQKFQGRVT
Heavy chain CDR2 region
(SEQ ID NO: 114)
LEWMGIINPRGDGTRYAQKFQGRVTM
Heavy chain CDR2 region
(SEQ ID NO: 115)
LEWMGIINPRGDGTRYAQKFQGRVTMT
Heavy chain CDR2 region
(SEQ ID NO: 116)
MGIINPRGDG
Heavy chain CDR2 region
(SEQ ID NO: 117)
MGIINPRGDGT
Heavy chain CDR2 region
(SEQ ID NO: 118)
MGIINPRGDGTR
Heavy chain CDR2 region
(SEQ ID NO: 119)
MGIINPRGDGTRY
Heavy chain CDR2 region
(SEQ ID NO: 120)
MGIINPRGDGTRYA
Heavy chain CDR2 region
(SEQ ID NO: 121)
MGIINPRGDGTRYAQ
Heavy chain CDR2 region
(SEQ ID NO: 122)
MGIINPRGDGTRYAQK
Heavy chain CDR2 region
(SEQ ID NO: 123)
MGIINPRGDGTRYAQKF
Heavy chain CDR2 region
(SEQ ID NO: 124)
MGIINPRGDGTRYAQKFQ
Heavy chain CDR2 region
(SEQ ID NO: 125)
MGIINPRGDGTRYAQKFQG
Heavy chain CDR2 region
(SEQ ID NO: 126)
MGIINPRGDGTRYAQKFQGR
Heavy chain CDR2 region
(SEQ ID NO: 127)
MGIINPRGDGTRYAQKFQGRV
Heavy chain CDR2 region
(SEQ ID NO: 128)
MGIINPRGDGTRYAQKFQGRVT
Heavy chain CDR2 region
(SEQ ID NO: 129)
MGIINPRGDGTRYAQKFQGRVTM
Heavy chain CDR2 region
(SEQ ID NO: 130)
MGIINPRGDGTRYAQKFQGRVTMT
Heavy chain CDR2 region
(SEQ ID NO: 131)
NPRGDG
Heavy chain CDR2 region
(SEQ ID NO: 132)
NPRGDGT
Heavy chain CDR2 region
(SEQ ID NO: 133)
NPRGDGTR
Heavy chain CDR2 region
(SEQ ID NO: 134)
NPRGDGTRY
Heavy chain CDR2 region
(SEQ ID NO: 135)
NPRGDGTRYA
Heavy chain CDR2 region
(SEQ ID NO: 136)
NPRGDGTRYAQ
Heavy chain CDR2 region
(SEQ ID NO: 137)
NPRGDGTRYAQK
Heavy chain CDR2 region
(SEQ ID NO: 138)
NPRGDGTRYAQKF
Heavy chain CDR2 region
(SEQ ID NO: 139)
NPRGDGTRYAQKFQ
Heavy chain CDR2 region
(SEQ ID NO: 140)
NPRGDGTRYAQKFQG
Heavy chain CDR2 region
(SEQ ID NO: 141)
NPRGDGTRYAQKFQGR
Heavy chain CDR2 region
(SEQ ID NO: 142)
NPRGDGTRYAQKFQGRV
Heavy chain CDR2 region
(SEQ ID NO: 143)
NPRGDGTRYAQKFQGRVT
Heavy chain CDR2 region
(SEQ ID NO: 144)
NPRGDGTRYAQKFQGRVTM
Heavy chain CDR2 region
(SEQ ID NO: 145)
NPRGDGTRYAQKFQGRVTMT
Heavy chain CDR2 region
(SEQ ID NO: 146)
PRGDGTRYAQ
Heavy chain CDR2 region
(SEQ ID NO: 147)
PRGDGTRYAQK
Heavy chain CDR2 region
(SEQ ID NO: 148)
PRGDGTRYAQKF
Heavy chain CDR2 region
(SEQ ID NO: 149)
PRGDGTRYAQKFQ
Heavy chain CDR2 region
(SEQ ID NO: 150)
PRGDGTRYAQKFQG
Heavy chain CDR2 region
(SEQ ID NO: 151)
PRGDGTRYAQKFQGR
Heavy chain CDR2 region
(SEQ ID NO: 152)
PRGDGTRYAQKFQGRV
Heavy chain CDR2 region
(SEQ ID NO: 153)
PRGDGTRYAQKFQGRVT
Heavy chain CDR2 region
(SEQ ID NO: 154)
PRGDGTRYAQKFQGRVTM
Heavy chain CDR2 region
(SEQ ID NO: 155)
PRGDGTRYAQKFQGRVTMT
Heavy chain CDR2 region
(SEQ ID NO: 156)
QGLEWMGIINP
Heavy chain CDR2 region
(SEQ ID NO: 157)
QGLEWMGIINPR
Heavy chain CDR2 region
(SEQ ID NO: 158)
QGLEWMGIINPRG
Heavy chain CDR2 region
(SEQ ID NO: 159)
QGLEWMGIINPRGD
Heavy chain CDR2 region
(SEQ ID NO: 160)
QGLEWMGIINPRGDG
Heavy chain CDR2 region
(SEQ ID NO: 161)
QGLEWMGIINPRGDGT
Heavy chain CDR2 region
(SEQ ID NO: 162)
QGLEWMGIINPRGDGTR
Heavy chain CDR2 region
(SEQ ID NO: 163)
QGLEWMGIINPRGDGTRY
Heavy chain CDR2 region
(SEQ ID NO: 164)
QGLEWMGIINPRGDGTRYA
Heavy chain CDR2 region
(SEQ ID NO: 165)
QGLEWMGIINPRGDGTRYAQ
Heavy chain CDR2 region
(SEQ ID NO: 166)
QGLEWMGIINPRGDGTRYAQK
Heavy chain CDR2 region
(SEQ ID NO: 167)
QGLEWMGIINPRGDGTRYAQKF
Heavy chain CDR2 region
(SEQ ID NO: 168)
RGDGTRYA
Heavy chain CDR2 region
(SEQ ID NO: 169)
RGDGTRYAQ
Heavy chain CDR2 region
(SEQ ID NO: 170)
RGDGTRYAQK
Heavy chain CDR2 region
(SEQ ID NO: 171)
RGDGTRYAQKF
Heavy chain CDR2 region
(SEQ ID NO: 172)
RGDGTRYAQKFQ
Heavy chain CDR2 region
(SEQ ID NO: 173)
RGDGTRYAQKFQG
Heavy chain CDR2 region
(SEQ ID NO: 174)
RGDGTRYAQKFQGR
Heavy chain CDR2 region
(SEQ ID NO: 175)
RGDGTRYAQKFQGRV
Heavy chain CDR2 region
(SEQ ID NO: 176)
RGDGTRYAQKFQGRVT
Heavy chain CDR2 region
(SEQ ID NO: 177)
RGDGTRYAQKFQGRVTM
Heavy chain CDR2 region
(SEQ ID NO: 178)
RGDGTRYAQKFQGRVTMT
Heavy chain CDR2 region
(SEQ ID NO: 179)
WMGIINPRGDG
Heavy chain CDR2 region
(SEQ ID NO: 180)
WMGIINPRGDGT
Heavy chain CDR2 region
(SEQ ID NO: 181)
WMGIINPRGDGTR
Heavy chain CDR2 region
(SEQ ID NO: 182)
WMGIINPRGDGTRY
Heavy chain CDR2 region
(SEQ ID NO: 183)
WMGIINPRGDGTRYA
Heavy chain CDR2 region
(SEQ ID NO: 184)
WMGIINPRGDGTRYAQ
Heavy chain CDR2 region
(SEQ ID NO: 185)
WMGIINPRGDGTRYAQK
Heavy chain CDR2 region
(SEQ ID NO: 186)
WMGIINPRGDGTRYAQKF
Heavy chain CDR2 region
(SEQ ID NO: 187)
WMGIINPRGDGTRYAQKFQ
Heavy chain CDR2 region
(SEQ ID NO: 188)
WMGIINPRGDGTRYAQKFQG
Heavy chain CDR2 region
(SEQ ID NO: 189)
WMGIINPRGDGTRYAQKFQGR
Heavy chain CDR2 region
(SEQ ID NO: 190)
WMGIINPRGDGTRYAQKFQGRV
Heavy chain CDR2 region
(SEQ ID NO: 191)
WMGIINPRGDGTRYAQKFQGRVT
Heavy chain CDR2 region
(SEQ ID NO: 192)
WMGIINPRGDGTRYAQKFQGRVTM
Heavy chain CDR2 region
(SEQ ID NO: 193)
WMGIINPRGDGTRYAQKFQGRVTMT
Heavy chain CDR1 region
(SEQ ID NO: 194)
YFYLH
Heavy chain CDR3 region
(SEQ ID NO: 195)
GADHGAFDI
Light chain CDR1 region
(SEQ ID NO: 196)
RASQSVRRNYFA
Light chain CDR2 region
(SEQ ID NO: 197)
DASTRAT
Light chain CDR3 region
(SEQ ID NO: 198)
QQYDSSPPMYI

### EXAMPLES

### Example 1: Intranasal Administration of Antibodies for the Treatment of SARS-CoV-2

The objective of this study is to compare the prophylactic efficacy of a single dose of a representative antibody according to claim 1, namely an antibody comprising a heavy chain variable domain that comprises a heavy chain CDR1 region of SEQ ID NO: 194, a heavy chain CDR2 region of SEQ ID NO: 082, and a heavy chain CDR3 region of SEQ ID NO: 195 and a light chain CDR1 region of SEQ ID NO: 196, a light chain CDR2 region of SEQ ID NO: 197, and a light chain CDR3 region of SEQ ID NO: 198, in a composition formulated with hypromellose (HPMC).

Animals (n=10 per group) are treated with an intranasal dose range of the test antibody (10 to 0.2 mg/kg) or PBS control at study day-1. At study day 0 animals are challenged with 25 LD50 of SARS-CoV-2 B.1.351 variant.

### Materials & Methods

The test item is a synthesized antibody according to claim 1.

The antibody is dissolved in DMSO and diluted with phosphate-buffered saline (PBS) to adjust the final concentration of DMSO to 10%, thereby preparing an antibody-DMSO-PBS solution containing a pharmaceutically acceptable excipient.

The antibody according to claim 1 is made up at various concentrations so that 100 μL administrations to a 20 g mouse will result in antibody doses ranging from 10 mg/kg, 5 mg/kg, 2.5 mg/kg, 0.5 mg/kg to 0.2 mg/kg.

The formulations are stored at −75° C.±10° C. The temperature is monitored. The virus strain tested is SARS-CoV-2 B.1.351 variant, obtainable from ATCC.

### Animals

The animal species and breed is Mouse (Mus musculus), SARS-CoV-2 Delta K18 hACE2 Tg mouse model or BALB/c AnNCrl, SPF at a weight of c. 16-18 g on the day of arrival at the facility. 60 mice are used at the age of 6-8 weeks on the day of arrival at the facility and are exclusively female. Ten animals are randomly allocated to 6 treatment groups. Potable water from the public supply is available ad libitum from water bottles. The animals have free access to feed (RMH-B from Altromin, Germany). Mice are maintained between a minimum and maximum temperature of 20.9° C. and 21.4° C. and at relative humidity percentage between 35 and 42%, under artificial lighting with 12 hours light and 12 hours dark.

### Study Design

The dose level of test antibody applied in the current example is based on a dose range proven to have prophylactic activity. A total number of 60 mice, 6 weeks of age at the time of arrival, are transported to the animal Facility and allocated to 6 experimental groups according to Table 1. Mice are given a period of 6 days for acclimatization. For studies with BALB/c AnNCrl mice, mice of 12 months of age are allocated to 6 experimental groups according to Table 1 (Zhang et al. Journal of Virology 95: e02477-20 2021).

TABLE 1
Treatment schedule

| Test | | Admin. | Dose | Day of | No. of |
|---|---|---|---|---|---|
| group | Antibody | route | (mg/kg) | admin. | Animals |
| 1 | SEQ ID NO: 082 | IN | 10 | −1 | 10 |
| 2 | SEQ ID NO: 082 | IN | 5 | −1 | 10 |
| 3 | SEQ ID NO: 082 | IN | 2.5 | −1 | 10 |
| 4 | SEQ ID NO: 082 | IN | 0.5 | −1 | 10 |
| 5 | SEQ ID NO: 082 | IN | 0.2 | −1 | 10 |
| 6 | Placebo PBS | IN | — | −1 | 10 |
| | | | | | 60 |

Female SARS-CoV-2 Delta K18 hACE2 Tg mouse model or BALB/c mice are treated via the intranasal route of administration with the antibody described above at a dose between 10 and 0.2 mg/kg based upon average mouse mass of 20 g. On Day 0, all mice are challenged with a lethal dose of SARS-CoV-2 B.1.351 variant and observed for clinical signs and weight loss until the end of the study at day 21.

### Antibody Administration

The test antibody described above is stored at −75° C.±10° C. upon arrival. The appropriate dose, according to the treatment schedule (Table 1), is formulated assuming the average mass of each mouse to be 20 g.

On the day of administration, the test antibody solutions are kept on ice during transfer to the animal facility. Just prior to dosing, the material is drawn into a syringe, allowed briefly to warm to room temperature and then administered to each mouse. Mice receive the indicated dose by intranasally administering 50 μL of antibody solution into the left and right nare (100 μL per mouse) using a pipette tip.

### Virus Administration

The virus material is stored at −75° C.±10° C. and is defrosted prior to administration. Once defrosted, the material is diluted in cold PBS corresponding to approximately 25 LD50 and kept on ice until administration to the mice. As required, the animals are anesthetized by intraperitoneal injection of a mixture of ketamine/xylazine and each animal receives approximately 50 μL of virus corresponding with approximately 3.1 log 10 TCID50 by intranasal inoculation using a pipette tip. Unused material is returned on ice to the lab for back titration along with the material that is kept on ice in the lab during inoculation of the animals.

### Laboratory Analysis

Laboratory analyses are performed in class II biological safety cabinets. Inoculum is returned to the lab and the actual dose of the virus administered is verified by titrating eight replicate samples on MDCK cells. Virus titres (TCID50/mL) are determined by the method of Reed and Münch.

### Clinical Monitoring

General health observations are performed on each animal from the day of arrival until the end of the study at least once daily (during normal servicing procedures). Clinical signs are scored from day −1 until the end of the study on day 21 using a scoring system (0=no clinical signs; 1=rough coat; 2=rough coat, less reactive during handling; 3=rough coat, rolled up, labored breathing, less reactive during handling; 4=rough coat, rolled up, labored breathing, inactive in response to manipulation/handlings). Animals are inspected twice a day as long as they received a score of 3 and are euthanized when they received a score of 4 (humane end point). Each animal is weighed daily beginning one day prior to infection (day −1). Calibrated weighing scales are used.

### Terminal Investigations

At the end of the study, on day 21, mice are euthanized by i.v. injection of Euthasol® (sodium pentobarbital) followed by cervical dislocation. Gross necropsy is not performed.

### Data Analysis and Statistical Methods

Survival proportions at day 21, survival times and change in bodyweight (Area Under the Curve) are compared to the corresponding control group using Fisher's exact test, log-rank and Welch's t-test, respectively. All groups are compared to the corresponding vehicle (DMSO-PBS solution) control group. Comparisons are performed using a stepwise approach within each treatment route (starting with the highest antibody dose conditionally testing a lower dose if the previous step is statistically significant). Statistical analysis is performed using R and statistical significance is set at α=0.05.

### Survival—Intranasal

Prophylactic treatment with ≥0.2 mg/kg test antibody is completely protective: 100% of the animals survived the viral challenge whereas the survival proportion at day 21 in the control group is 0%. The median survival time of the control group is 7 days.

Survival times of the test antibody treated groups are compared to the control group using a log-rank test. Prophylactic treatment with ≥0.2 mg/kg test antibody results in a significant improvement in survival time.

### Body Weight

Change in bodyweight is analysed using an Area Under the Curve (AUC) analysis in which the last observed body weight is carried forward if a mouse died/is euthanized during the study. The weight per mouse at day 0 is used as baseline and weight change is determined relative to baseline. The net AUC's is compared using a Welch's t-test due to unequal variances. Area under the curve results, for the intranasal prophylactic treatment group are determined.

### Intranasal

Prophylactic treatment with ≥0.2 mg/kg test antibody results in a significant reduction in weight loss compared to the control group.

No clinical signs are recorded in the groups treated with ≥0.2 mg/kg test an antibody. All animals survived the challenge with SARS-CoV-2 B.1.351 variant

### Clinical Score

Median Clinical Scores, for the intranasal group is determined.

### CONCLUSION

In this lethal SARS-CoV-2 B.1.351 variant mouse model, the prophylactic intranasal administration of ≥0.2 mg/kg test antibody provides 100% survival and a reduction in weight loss, whereas all the animals in the control group die.

1. A composition comprising an antibody comprising a heavy chain variable domain that comprises a heavy chain CDR1 region of SEQ ID NO: 194, a heavy chain CDR2 region of SEQ ID NO: 082, and a heavy chain CDR3 region of SEQ ID NO: 195 and a light chain CDR1 region of SEQ ID NO: 196, a light chain CDR2 region of SEQ ID NO: 197, and a light chain CDR3 region of SEQ ID NO: 198 and a pharmaceutically acceptable mucoadhesive excipient.

2. A composition according to claim 1 wherein the pharmaceutically acceptable mucoadhesive excipient comprises any one of mucoadhesive polymers, cellulose derivatives, preferably hypromellose (HPMC).

3. A composition according to claim 1 or 2 wherein the amino acid sequence has at least 80%, 81%, 82%, 83%, 84%, 85%, 86%, 87%, 88%, 89%, 90%, 91%, 92%, 93%, 94%, 95%, 96%, 97%, 98%, or at least 99% sequence identity.

4. A composition according to any one of the preceding claims, for use in a method for treatment of a betacoronavirus infection, preferably a SARS-CoV-2 virus infection or variants of the SARS-CoV-2 virus in an individual, wherein the antibody is administered intranasally and/or by oral inhalation.

5. A composition for use according to any one of the preceding claims, wherein the composition is for the treatment of SARS-CoV-2 virus infection as prophylactic and/or therapeutic treatment of SARS-CoV-2 virus infection.

6. A composition for use according to any one of the preceding claims, wherein the heavy chain CDR2 region comprises at most any one of 1, 2 or 3 amino acid insertions, deletions or substitutions.

7. A composition for use according to any one of the preceding claims, wherein the antibody is administered intranasally.

8. A composition for use according to any one of the preceding claims, wherein between 0.01 μg and 20 mg of the antibody is administered to an individual.

9. A composition for use according to any one of the preceding claims, wherein the antibody is administered at least once or at least twice per day, preferably wherein the antibody is administered at least once or at least twice per week.

10. A composition for use according to any one of the preceding claims, wherein the composition is formulated for intranasal administration or oral inhalation comprising an antibody according to any one of the preceding claims, in a single dose unit of between 0.01 μg and 20 mg.

11. The composition according to claim 10 wherein said antibody is carried by a Lipid Nano-Particle (LNP), a liposome, or a virus-like particle (VLP).

12. A nucleic acid molecule encoding the antibody according to any one of the preceding claims.

13. An expression vector comprising the nucleic acid molecule of claim 12.

14. A host cell comprising the nucleic acid molecule of claim 12, or the expression vector of claim 13.

15. A method of preparing a composition according to any one of the preceding claims, comprising:
a) introducing the expression vector of claim 13 into the host cells of claim 14;
b) culturing the host cells in a culture medium, optionally comprising Isopropyl β-D-1-thiogalactopyranoside (IPTG) under conditions allowing the expression of the antibody in said host cells;
c) lysing the host cells;
d) separating the lysed cells, optionally into an insoluble fraction and a soluble fraction;
e) solubilizing the lysed cells, optionally into an insoluble fraction using an extraction buffer, optionally comprising a chaotrophic agent and a reducing agent; and optionally
f) dialyzing the solution obtained in step (e) in the presence of arginine at concentrations from 0.5M to 2M, preferably from 0.6M to 1.5M, preferably from 0.7M to 1M, thereby obtaining isolated antibodies.
